(19) United States
(12) Patent Application Publication
(10) Pub. No.: US 2006/0147478 A1
(43) Pub. Date: Jul. 6, 2006

(54) Protein involved in carcinoma

(76) Inventor: Jonathan Alexander Terrett, Berkshire (GB)

Correspondence Address: KLAUBER & JACKSON, 411 HACKENSACK AVENUE, HACKENSACK, NJ, 07601, US

(21) Appl. No.: 10/524,910

(22) PCT Filed: Aug. 6, 2003
(86) PCT No.: PCT/GB03/03421

§ 371 Date: Jan. 12, 2006

(30) Foreign Application Priority Data
Aug. 24, 2002 (GB) 0219776.2

(51) Int. Cl. C12Q 1/68; C07H 21/04; C12P 21/06; A61K 39/395; A61K 39/00

(52) U.S. Cl. 424/277.1; 424/155.1; 435/6; 435/69.1; 435/320.1; 435/325; 530/350; 530/388.8; 536/23.5

(57) ABSTRACT

The present invention provides a polypeptide, PTK7, of use in the treatment and/or prophylaxis of carcinoma. Also provided are agents which interact with or modulate the expression or activity of the polypeptide, methods for the identification of such agents and the use of PTK7 in the diagnosis of carcinoma.

The present invention relates to methods for the treatment and/or prophylaxis of carcinoma comprising targeting of the polypeptide, PTK7, agents which interact with or modulate the expression or activity of the polypeptide, methods for the identification of such agents and the use of PTK7 in the diagnosis of carcinoma.

Tumour specific proteins have been identified for a number of cancer types using techniques such as differential screening of cDNAs (Hubert, R. S., et al., 1999, Proc. Natl. Acad. Sci. USA 96:14523-14528) and the purification of cell-surface proteins that are recognised by tumour-specific antibodies (Catimel, B., et al., 1996, J. Biol. Chem. 271: 25664-25670). More recently, DNA ‘chips’ containing up to 10,000 expressed sequence elements have been used to characterise tumour cell gene expression (Dhanasekaran, S. M., et al., 2001, Nature 412:822-826). However, there are several reasons why the numerous and extensive previous transcriptomic analysis of cancers may not have revealed all, or even most, tumour associated proteins. These include: (i) a lack of correlation between transcript and disease-associated protein levels, particularly common for membrane proteins that often have a long half-life and as such do not have a high mRNA turnover. Therefore, whilst the difference in protein levels between normal and cancerous cells are consistent it is often difficult to associate changes in the mRNA for a given membrane protein with the cancerous state. (ii) Translocation of a protein in the disease state rather than simply differential levels of the transcript, for example, erbB2/HER2-neu, shows much greater plasma-membrane localisation in cancer cells than normal breast cells, and the transcription factors oestrogen receptor and STAT3 translocate to the nucleus to exert their tumourigenic effects. (iii) Novel, uncharacterised genes are not highly represented within the ‘closed system’ of a cDNA array where there are restrictions on the number of expressed sequence elements per chip and the knowledge and availability of DNA clones. It is well established that there is an unreliable relationship between protein expression and mRNA levels (e.g. Gygi S P et al., Mol. Cell Biol. 1999, 19:1720-30) as protein expression is subject to strict translational control at several levels. Regulation of the overall activity of the translational apparatus of a cell is expected to affect the translation of essentially all mRNAs (Matthews, M. et al., in Translational Control by Hershey, J. et al., pp 11-12, Cold Spring Harbour laboratory Press, 1996). Indeed, a fraction of specific mRNA is completely repressed. Furthermore individual mRNAs differ greatly in their efficiencies of translation and can be ‘weak’ or ‘strong’, thus contributing to the regulation of gene expression. Thus, the existence of a conceptual translation of a cDNA cannot provide definitive evidence of the existence of a particular protein in a particular cell type.

Breast cancer is the most frequently diagnosed cancer in women. The implementation of screening programs for the early detection of breast cancer, and the advent of anticancer treatments, such as chemotherapy, radiotherapy and anti-oestrogen therapies, to augment surgical resection have improved the survival of breast cancer patients. However, some breast tumours become refractory to such treatments, as the cancer cells develop resistance to chemotherapy drugs or lose their hormone sensitivity, leading to recurrent or metastatic disease which is often incurable. More recently, attention has focussed on the development of immunological therapies (Green, M C. et al., 2000, Cancer Treat. Rev. 26:269-286; Davis, I D., 2000, Immunol. Cell Biol. 78:179-195; Knuth, A. et al., 2000, Cancer Chemother Pharmacol. 46:S46-51; Shiku, H. et al., 2000, Cancer Chemother. Pharmacol. 46:S77-82; Saffran, D C. et al., 1999, Cancer Metastasis Rev. 18:437-449), such as cancer vaccines and monoclonal antibodies (mAbs), as a means of initiating and targeting a host immune response against tumour cells. Herceptin, a mAb that recognises the erbB2/HER2-neu receptor protein, is used as a treatment for metastatic breast cancer. In combination with chemotherapy, Herceptin has been shown to prolong the time to disease progression, when compared to patients receiving chemotherapy alone (Baselga, J. et al., 1998, Cancer Res. 58:2825-2831). Herceptin, however, is only effective in treating the 10-20% of patients whose tumours over-express the erbB2 protein.

Pancreatic cancer treatments include therapeutic monoclonal antibodies; for example, a combination treatment is currently in clinical trials involving a combination of a monoclonal antibody, IMC-C225, directed against the epidermal growth factor (EGF) receptor (the EGF receptor is over-expressed in pancreatic cancer tumors), and 5-fluorouracil (5-FU). It has also been reported that the HER-2/neu oncogene was over-expressed in 21% of 154 patients with pancreatic adenocarcinoma (Safran, H. et al., 2001 Amer. J. Clin. Oncology 24:496-499) where it has been suggested that therapeutic evaluation of recombinant humanized anti-HER2 antibody (Herceptin) with such patients may be warranted. Diagnosis of pancreatic cancer is currently difficult as early symptoms are similar to those of other disorders including chronic pancreatitis, hepatitis, gall stones and diabetes mellitus. Often, by the time a correct diagnosis has been made, the cancer has spread to the lymph nodes and the liver.

Bladder cancer is a disease which occurs more frequently in men than women and is often diagnosed at an advanced stage. Treatment is generally restricted to surgery, radiotherapy and chemotherapy with few biological/immunological treatments available at the present time. In particular, BCG (Bacillus of Calmette and Guerin), has been used to stimulate the immune system of patients with bladder cancer.

Cancer of the kidney, sometimes referred to as renal cell carcinoma, is the most common tumour rising from the kidney. At present, surgery is the main treatment for kidney cancer with radiotherapy being sometimes used instead of surgery for patients who are too ill to undergo a major operation, however, renal cell kidney cancers are not particularly sensitive to radiotherapy. The two most common biological treatments for kidney cancer are the use of the cytokines, interleukin-2 and interferon-alpha, which are used in an attempt to stimulate the immune system of the patient.

Lung cancer is a leading cancer killer in both men and women with two major types: non-small cell lung cancer and small cell lung cancer. Treatment is restricted to surgery, where possible, chemotherapy and radiotherapy.

Ovarian cancer is the deadliest of the gynaecological cancers with around 70% of sufferers with the more common epithelial ovarian cancer initially presenting with late stage disease. Their survival rate is significantly reduced compared to those who present with earlier stage disease because the cancer will have spread to the upper abdomen. Ovarian cancer has been generally treated with cisplatin-based chemotherapy and often recurs due to acquired cisplatin resistance (Yahata, H. et al., 2002, J. Cancer Res. Clin. Oncol. 128:621-6), hence the need for new drugs and new therapeutic targets. There is also a need for new markers of ovarian cancer as current markers lack adequate sensitivity and specificity to be applicable in large populations (Rai, A. et al., 2002, Arch. Pathol. Lab. Med. 126:1518-26).

Osteosarcoma is primarily a childhood disease characterised by bone lesions. More than 20% of patients are diagnosed with late stage osteosarcoma with definitive diagnosis most often via biopsy. Treatment is generally chemotherapy combined with surgery.

Thus, important needs exist for new therapeutic agents for the treatment of carcinoma. Additionally, there is a clear need to identify new carcinoma associated proteins for use as sensitive and specific biomarkers for the diagnosis of carcinoma in living subjects.

cDNA encoding PTK-7, also known as CCK-4, was first isolated by Lee et al., (1993, Oncogene 8:3403-3410) and later by Mossie et al., (WO 96/37610 and Oncogene 1995, 11:2179-2184). In contrast to our findings the latter authors found PTK7 mRNA expression to be absent in normal colon tissue. PTK7 is a new member of the receptor tyrosine kinase family.

The present invention is based on the finding that PTK7 represents a novel therapeutic target for the treatment and/or prophylaxis of carcinomas.

Accordingly, the invention provides a method for the treatment and/or prophylaxis of carcinoma comprising administering a therapeutically effective amount of an agent which interacts with or modulates the expression or activity of a PTK7 polypeptide.

A PTK7 polypeptide includes a polypeptide which:

(a) comprises or consists of the amino acid sequence of SEQ ID NO:1; or

(b) is a derivative having one or more amino acid substitutions, modifications, deletions or insertions relative to the amino acid sequence of SEQ ID NO:1 which retains the activity of the PTK7 polypeptide.

The term “polypeptides” includes peptides, polypeptides and proteins. These are used interchangeably unless otherwise specified.

In the present application, the term “carcinoma” includes a malignant new growth that arises from epithelium, found in skin or, more commonly, the lining of body organs, for example: breast, lung, kidney, pancreas, ovary, prostate, bladder, stomach or bowel. Osteosarcoma is also included. Carcinomas tend to infiltrate into adjacent tissue and spread (metastasise) to distant organs, for example: to bone, liver, lung or the brain. In the methods of the invention the carcinoma is preferably ovarian cancer or osteosarcoma, more preferably lung, kidney, pancreatic or bladder cancer and most preferably breast cancer and in particular metastatic breast cancers.

Agents of use in the methods of the invention include without limitation, agents that are capable of interacting with (e.g. binding to, or recognising) a PTK7 polypeptide or a nucleic acid molecule encoding a PTK7 polypeptide, or are capable of modulating the interaction, expression or activity of a PTK7 polypeptide or the expression of a nucleic acid molecule encoding a PTK7 polypeptide. The skilled person will therefore appreciate that the methods of the invention include direct targeting of the PTK7 polypeptide as well as indirectly affecting the expression of the polypeptide by targeting the corresponding nucleic acid. Preferably, a PTK7 polypeptide is targeted directly. Such agents include, without limitation, antibodies, nucleic acids (e.g. DNA and RNA), carbohydrates, lipids, proteins, polypeptides, peptides, peptidomimetics, small molecules and other drugs.

Thus, the invention also provides the use of an agent, which interacts or modulates the expression or activity of a PTK7 polypeptide in the manufacture of a medicament for the treatment and/or prophylaxis of carcinoma.

Most preferably, the agent for use in the prophylaxis and/or treatment of carcinoma is an antibody which interacts with (i.e. binds to or recognises) or modulates the activity of a PTK7 polypeptide. Further preferred are antibodies which specifically recognise a PTK7 polypeptide. Specifically recognising or binding specifically means that the antibodies have a greater affinity for PTK7 polypeptides than for other polypeptides.

Accordingly, there is provided the use of an antibody which specifically recognises a PTK7 polypeptide for use in the manufacture of a medicament for use in the treatment and/or prophylaxis of carcinoma. Also provided is a method of treatment and/or prophylaxis of carcinoma in a subject comprising administering to said subject a therapeutically effective amount of an antibody which specifically recognises PTK7. In particular, an antibody which specifically interacts with a PTK7 polypeptide may be used to mediate antibody dependent cell cytotoxicity (ADCC) and complement dependent cytotoxicity (CDC). In such a case the antibody is preferably a full length naked antibody. In another aspect of the invention, antibodies which specifically bind to PTK7 polypeptides may be used to inhibit the activity of said polypeptides.

An antibody, optionally conjugated to a therapeutic moiety, can be used therapeutically alone or in combination with a cytotoxic factor(s) and/or cytokine(s). In particular, PTK7 antibodies can be conjugated to a therapeutic agent, such as a cytotoxic agent, a radionuclide or drug moiety to modify a given biological response. The therapeutic agent is not to be construed as limited to classical chemical therapeutic agents. For example, the therapeutic agent may be a drug moiety which may be a protein or polypeptide possessing a desired biological activity. Such moieties may include, for example and without limitation, a toxin such as abrin, ricin A, pseudomonas exotoxin, or diphtheria toxin, a protein such as tumour necrosis factor, α-interferon, β-interferon, nerve growth factor, platelet derived growth factor or tissue plasminogen activator, a thrombotic agent or an anti-angiogenic agent, e.g. angiostatin or endostatin, or, a biological response modifier such as a lymphokine, interleukin-1 (IL-1), interleukin-2 (IL-2), interleukin-6 (IL-6), granulocyte macrophage colony stimulating factor (GM-CSF), granulocyte colony stimulating factor (G-CSF), nerve growth factor (NGF) or other growth factor.

Therapeutic agents also include cytotoxins or cytotoxic agents including any agent that is detrimental to (e.g. kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents also include, but are not limited to, antimetabolites (e.g. methotrexate, 6-mercaptopurine, 6-thioguanine, cytarabine, 5-fluorouracil decarbazine), alkylating agents (e.g. mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g. daunorubicin (formerly daunomycin) and doxorubicin), antibiotics (e.g. dactinomycin (formerly actinomycin), bleomycin, mithramycin, anthramycin (AMC), calicheamicins or duocarmycins), and anti-mitotic agents (e.g. vincristine and vinblastine).

Other therapeutic moieties may include radionuclides such as ¹¹¹In and ⁹⁰Y, Lu¹⁷⁷, Bismuth²¹³, Californium²⁵², Iridium¹⁹² and Tunsten¹⁸⁸/Rhenium¹⁸⁸; or drugs such as but not limited to, alkylphosphocholines, topoisomerase I inhibitors, taxoids and suramin.

Techniques for conjugating such therapeutic agents to antibodies are well known in the art (see, e.g. Arnon et al., “Monoclonal Antibodies For Immunotargeting Of Drugs In Cancer Therapy”, in Monoclonal Antibodies And Cancer Therapy, Reisfeld et al., eds., 1985 pp. 243-56, ed. Alan R. Liss, Inc; Hellstrom et al., “Antibodies For Drug Delivery”, in Controlled Drug Delivery, 2nd Ed., Robinson et al., eds., 1987, pp. 623-53, Marcel Dekker, Inc.; Thorpe, “Antibody Carriers Of Cytotoxic Agents In Cancer Therapy: A Review”, in Monoclonal Antibodies '84: Biological And Clinical Applications; Pinchera et al., 1985, eds., pp. 475-506; “Analysis, Results, And Future Prospective Of The Therapeutic Use Of Radiolabelled Antibody In Cancer Therapy”, in Monoclonal Antibodies For Cancer Detection And Therapy, Baldwin et al. (eds.), 1985, pp. 303-16, Academic Press; Thorpe et al., 1982 “The Preparation And Cytotoxic Properties Of Antibody-Toxin Conjugates”, Immunol. Rev., 62:119-58 and Dubowchik et al., 1999, Pharmacology and Therapeutics, 83, 67-123). The antibodies for use in the invention include analogues and derivatives that are modified, for example but without limitation, by the covalent attachment of any type of molecule. Preferably, said attachment does not impair immunospecific binding. In one aspect, an antibody can be conjugated to a second antibody to form an antibody heteroconjugate (see U.S. Pat. No. 4,676,980).

In other embodiments, the invention provides the therapeutic use of fusion proteins of the antibodies (or functionally active fragments thereof), for example but without limitation, where the antibody or fragment thereof is fused via a covalent bond (e.g. a peptide bond), at optionally the N-terminus or the C-terminus, to an amino acid sequence of another protein (or portion thereof; preferably at least a 10, 20 or 50 amino acid portion of the protein). Preferably the antibody, or fragment thereof, is linked to the other protein at the N-terminus of the constant domain of the antibody. In another aspect, an antibody fusion protein may facilitate depletion or purification of a polypeptide as described herein, increase half-life in vivo, and enhance the delivery of an antigen across an epithelial barrier to the immune system.

Where the fusion protein is an antibody fragment linked to an effector or reporter molecule, this may be prepared by standard chemical or recombinant DNA procedures in which the antibody fragment is linked either directly or via a coupling agent to the effector or reporter molecule either before or after reaction with the activated polymer as appropriate. Particular chemical procedures include, for example, those described in WO 93/62331, WO 92/22583, WO 90,195 and WO 89/1476. Alternatively, where the effector or reporter molecule is a protein or polypeptide the linkage may be achieved using recombinant DNA procedures, for example as described in WO 86/01533 and EP 0392745.

Most preferably antibodies are attached to poly(ethyleneglycol) (PEG) moieties. Preferably, a modified Fab fragment is PEGylated, i.e. has PEG (poly(ethyleneglycol)) covalently attached thereto, e.g. according to the method disclosed in EP-A-0948544 [see also “Poly(ethyleneglycol) Chemistry, Biotechnical and Biomedical Applications”, 1992, J. Milton Harris (ed), Plenum Press, New York, “Poly(ethyleneglycol) Chemistry and Biological Applications”, 1997, J. Milton Harris and S. Zalipsky (eds), American Chemical Society, Washington D.C. and “Bioconjugation Protein Coupling Techniques for the Biomedical Sciences”, 1998, M. Aslam and A. Dent, Grove Publishers, New York; Chapman, A. 2002, Advanced Drug Delivery Reviews 2002, 54:531-545]. In one embodiment, a PEG modified Fab fragment has a maleimide group covalently linked to a single thiol group in a modified hinge region. A lysine residue may be covalently linked to the maleimide group. To each of the amine groups on the lysine residue may be attached a methoxypoly(ethyleneglycol) polymer having a molecular weight of approximately 20,000 Da. The total molecular weight of the entire effector molecule may therefore be approximately 40,000 Da.

PTK7 polypeptides or cells expressing said polypeptides can be used to produce antibodies, e.g. which specifically recognise said PTK7 polypeptides. Antibodies generated against a PTK7 polypeptide may be obtained by administering the polypeptides to an animal, preferably a non-human animal, using well-known and routine protocols.

Anti-PTK7 antibodies include functionally active fragments, derivatives or analogues and may be, but are not limited to, polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. Humanized antibodies are antibody molecules from non-human species having one or more complementarity determining regions (CDRs) from the non-human species and a framework region from a human immunoglobulin molecule (see, e.g. U.S. Pat. No. 5,585,089). Antibodies include immunoglobulin molecules and immunologically active portions of immunoglobulin molecules, i.e. molecules that contain an antigen binding site that specifically binds an antigen. The immunoglobulin molecules of the invention can be of any class (e.g. IgG, IgE, IgM, IgD and IgA) or subclass of immunoglobulin molecule.

Monoclonal antibodies may be prepared by any method known in the art such the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985).

Chimeric antibodies are those antibodies encoded by immunoglobulin genes that have been genetically engineered so that the light and heavy chain genes are composed of immunoglobulin gene segments belonging to different species. These chimeric antibodies are likely to be less antigenic. Bispecific antibodies may be made by methods known in the art (Milstein et al., 1983, Nature 305:537-539; WO 93/08829, Traunecker et al., 1991, EMBO J. 10:3655-3659).

The antibodies for use in the invention may be generated using single lymphocyte antibody methods based on the molecular cloning and expression of immunoglobulin variable region cDNAs generated from single lymphocytes that were selected for the production of specific antibodies such as described by Babcook, J. et al., 1996, Proc. Natl. Acad. Sci. USA 93(15):7843-7848 and in WO92/02551.

The antibodies for use in the present invention can also be generated using various phage display methods known in the art and include those disclosed by Brinkman et al. (in J. Immunol. Methods, 1995, 182:41-50), Ames et al. (J. Immunol. Methods, 1995, 184:177-186), Kettleborough et al. (Eur. J. Immunol. 1994, 24:952-958), Persic et al. (Gene, 1997 187 9-18), Burton et al. (Advances in Immunology, 1994,57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108. Techniques for the production of single chain antibodies, such as those described in U.S. Pat. No. 4,946,778 can also be adapted to produce single chain antibodies to PTK7 polypeptides. Also, transgenic mice, or other organisms, including other mammals, may be used to express humanized antibodies.

PTK7 polypeptides can be used for the identification of agents for use in the methods of treatment and/or prophylaxis according to the invention.

A further aspect of the invention provides methods of screening for anti-carcinoma agents that interact with a PTK7 polypeptide comprising:

(a) contacting said polypeptide with a candidate agent; and

(b) determining whether or not the candidate agent interacts with said polypeptide.

Preferably, the determination of an interaction between the candidate agent and PTK7 polypeptide comprises quantitatively detecting binding of the candidate agent and said polypeptide.

Further provided is a method of screening for anti-carcinoma agents that modulate the expression or activity of a PTK7 polypeptide comprising:
(i) comparing the expression or activity of said polypeptide in the presence of a candidate agent with the expression or activity of said polypeptide in the absence of the candidate agent or in the presence of a control agent; and
(ii) determining whether the candidate agent causes the expression or activity of said polypeptide to change.

Preferably, the expression and/or activity of a PTK7 polypeptide is compared with a predetermined reference range or control.

More preferably the method further comprises selecting an agent, which interacts with a PTK7 polypeptide or is capable of modulating the interaction, expression or activity of a PTK7 polypeptide, for further testing for use in the treatment and/or prophylaxis of carcinoma. It will be apparent to one skilled in the art that the above screening methods are also appropriate for screening for anti-carcinoma agents which interact with or modulate the expression or activity of a PTK7 nucleic acid molecule.

The invention also provides assays for use in drug discovery in order to identify or verify the efficacy of agents for treatment or prophylaxis of carcinoma. Agents identified using these methods can be used as lead agents for drug discovery, or used therapeutically. Expression of a PTK7 polypeptide can be assayed by, for example, immunoassays, gel electrophoresis followed by visualisation, detection of mRNA or PTK7 polypeptide activity, or any other method taught herein or known to those skilled in the art. Such assays can be used to screen candidate agents, in clinical monitoring or in drug development.

Agents can be selected from a wide variety of candidate agents. Examples of candidate agents include but are not limited to, nucleic acids (e.g. DNA and RNA), carbohydrates, lipids, proteins, polypeptides, peptides, peptidomimetics, small molecules and other drugs. Agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is suited to peptide libraries, while the other four approaches are applicable to peptide, non-peptide oligomer or small molecule libraries of compounds (Lam, 1997, Anticancer Drug Des. 12:145; U.S. Pat. No. 5,738,996; and U.S. Pat. No. 5,807,683).

Examples of suitable methods based on the present description for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented, for example, in solution (e.g. Houghten, 1992, Bio/Techniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et al., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

In one embodiment, agents that interact with (e.g. bind to) a PTK7 polypeptide are identified in a cell-based assay where a population of cells expressing a PTK7 polypeptide is contacted with a candidate agent and the ability of the candidate agent to interact with the polypeptide is determined. Preferably, the ability of a candidate agent to interact with a PTK7 polypeptide is compared to a reference range or control. In another embodiment, a first and second population of cells expressing a PTK7 polypeptide are contacted with a candidate agent or a control agent and the ability of the candidate agent to interact with the polypeptide is determined by comparing the difference in interaction between the candidate agent and control agent. If desired, this type of assay may be used to screen a plurality (e.g. a library) of candidate agents using a plurality of cell populations expressing a PTK7 polypeptide. If desired, this assay may be used to screen a plurality (e.g. a library) of candidate agents. The cell, for example, can be of prokaryotic origin (e.g. E. coli) or eukaryotic origin (e.g. yeast or mammalian). Further, the cells can express the PTK7 polypeptide endogenously or be genetically engineered to express the polypeptide. In some embodiments, a PTK7 polypeptide or the candidate agent is labelled, for example with a radioactive label (such as ³²P, ³⁵S or ¹²⁵I) or a fluorescent label (such as fluorescein isothiocyanate, rhodamine, phycoerythrin, phycocyanin, allophycocyanin, o-phthaldehyde or fluorescamine) to enable detection of an interaction between a polypeptide and a candidate agent.

In another embodiment, agents that interact with (e.g. bind to) a PTK7 polypeptide are identified in a cell-free assay system where a sample expressing a PTK7 polypeptide is contacted with a candidate agent and the ability of the candidate agent to interact with the polypeptide is determined. Preferably, the ability of a candidate agent to interact with a PTK7 polypeptide is compared to a reference range or control. In a preferred embodiment, a first and second sample comprising native or recombinant PTK7 polypeptide are contacted with a candidate agent or a control agent and the ability of the candidate agent to interact with the polypeptide is determined by comparing the difference in interaction between the candidate agent and control agent. If desired, this assay may be used to screen a plurality (e.g. a library) of candidate agents using a plurality of PTK7 polypeptide samples. Preferably, the polypeptide is first immobilized, by, for example, contacting the polypeptide with an immobilized antibody which specifically recognizes and binds it, or by contacting a purified preparation of polypeptide with a surface designed to bind proteins. The polypeptide may be partially or completely purified (e.g. partially or completely free of other polypeptides) or part of a cell lysate. Further, the polypeptide may be a fusion protein comprising the PTK7 polypeptide or a biologically active portion thereof and a domain such as glutathionine-S-transferase. Alternatively, the polypeptide can be biotinylated using techniques well known to those of skill in the art (e.g. biotinylation kit, Pierce Chemicals; Rockford, Ill.). The ability of the candidate agent to interact with the polypeptide can be duplicated by methods known to those of skill in the art.

In one embodiment, a PTK7 polypeptide is used as a “bait protein” in a two-hybrid assay or three hybrid assay to identify other proteins that bind to or interact with the PTK7 polypeptide (see e.g. U.S. Pat. No. 5,283,317; Zervos et al., 1993, Cell 72:223-232; Madura et al. 1993, J. Biol. Chem. 268:12046-12054; Bartel et al., 1993, Bio/Techniques 14:920-924; Iwabuchi et al., 1993, Oncogene 8:1693-1696; and WO 94/10300). As those skilled in the art will appreciate, such binding proteins are also likely to be involved in the propagation of signals by a PTK7 polypeptide. For example, they may be upstream or downstream elements of a signalling pathway involving a PTK7 polypeptide. Alternatively, polypeptides that interact with a PTK7 polypeptide can be identified by isolating a protein complex comprising a PTK7 polypeptide (said polypeptide may interact directly or indirectly with one or more other polypeptides) and identifying the associated proteins using methods known in the art such as mass spectrometry or Western blotting (for examples see Blackstock, W. & Weir, M. 1999, Trends in Biotechnology, 17:121-127; Rigaut, G. 1999, Nature Biotechnology, 17:1030-1032; Husi, H. 2000, Nature Neurosci. 3:661-669; Ho, Y. et al., 2002, Nature, 415:180-183; Gavin, A. et al., 2002, Nature, 415:141-147).

In all cases, the ability of the candidate agent to interact directly or indirectly with the PTK7 polypeptide can be determined by methods known to those of skill in the art. For example but without limitation, the interaction between a candidate agent and a PTK7 polypeptide can be determined by flow cytometry, a scintillation assay, an activity assay, mass spectrometry, microscopy, immunoprecipitation or western blot analysis.

In yet another embodiment, agents that competitively interact with (i.e. competitively binding to) a PTK7 polypeptide are identified in a competitive binding assay and the ability of the candidate agent to interact with the PTK7 polypeptide is determined. Preferably, the ability of a candidate agent to interact with a PTK7 polypeptide is compared to a reference range or control. In a preferred embodiment, a first and second population of cells expressing both a PTK7 polypeptide and a protein which is known to interact with the PTK7 polypeptide are contacted with a candidate agent or a control agent. The ability of the candidate agent to competitively interact with the PTK7 polypeptide is then determined by comparing the interaction in the first and second population of cells. In another embodiment, an alternative second population or a further population of cells may be contacted with an agent which is known to competitively interact with a PTK7 polypeptide. Alternatively, agents that competitively interact with a PTK7 polypeptide are identified in a cell-free assay system by contacting a first and second sample comprising a PTK7 polypeptide and a protein known to interact with the PTK7 polypeptide with a candidate agent or a control agent. The ability of the candidate agent to competitively interact with the PTK7 polypeptide is then determined by comparing the interaction in the first and second sample. In another embodiment, an alternative second sample or a further sample comprising a PTK7 polypeptide may be contacted with an agent which is known to competitively interact with a PTK7 polypeptide. In any case, the PTK7 polypeptide and known interacting protein may be expressed naturally or may be recombinantly expressed; the candidate agent may be added exogenously, or be expressed naturally or recombinantly.

In another embodiment, agents that modulate the interaction between a PTK7 polypeptide and another agent, for example but without limitation a protein, may be identified in a cell-based assay by contacting cells expressing a PTK7 polypeptide in the presence of a known interacting agent and a candidate modulating agent and selecting the candidate agent which modulates the interaction. Alternatively, agents that modulate an interaction between a PTK7 polypeptide and another agent, for example but without limitation a protein, may be identified in a cell-free assay system by contacting the polypeptide with an agent known to interact with the polypeptide in the presence of a candidate agent. A modulating agent can act as an antibody, a cofactor, an inhibitor, an activator or have an antagonistic or agonistic effect on the interaction between a PTK7 polypeptide and a known agent. As stated above the ability of the known agent to interact with a PTK7 polypeptide can be determined by methods known in the art. These assays, whether cell-based or cell-free, can be used to screen a plurality (e.g. a library) of candidate agents.

In another embodiment, a cell-based assay system is used to identify agents capable of modulating (i.e. stimulating or inhibiting) the activity of a PTK7 polypeptide. Accordingly, the activity of a PTK7 polypeptide is measured in a population of cells that naturally or recombinantly express a PTK7 polypeptide, in the presence of a candidate agent. Preferably, the activity of a PTK7 polypeptide is compared to a reference range or control. In a preferred embodiment, the activity of a PTK7 polypeptide is measured in a first and second population of cells that naturally or recombinantly express a PTK7 polypeptide, in the presence of agent or absence of a candidate agent (e.g. in the presence of a control agent) and the activity of the PTK7 polypeptide is compared. The candidate agent can then be identified as a modulator of the activity of a PTK7 polypeptide based on this comparison. Alternatively, the activity of a PTK7 polypeptide can be measured in a cell-free assay system where the PTK7 polypeptide is either natural or recombinant. Preferably, the activity of a PTK7 polypeptide is compared to a reference range or control. In a preferred embodiment, the activity of a PTK7 polypeptide is measured in a first and second sample in the presence or absence of a candidate agent and the activity of the PTK7 polypeptide is compared. The candidate agent can then be identified as a modulator of the activity of a PTK7 polypeptide based on this comparison.

The activity of a PTK7 polypeptide can be assessed by detecting its effect on a downstream effector, for example but without limitation, the level or activity of a second messenger (e.g. cAMP, intracellular Ca²⁺, diacylglycerol, IP₃, etc.), detecting catalytic or enzymatic activity, detecting the induction of a reporter gene (e.g. luciferase) or detecting a cellular response, for example, proliferation, differentiation or transformation where appropriate as known by those skilled in the art (for activity measurement techniques see, e.g. U.S. Pat. No. 5,401,639). The candidate agent can then be identified as a modulator of the activity of a PTK7 polypeptide by comparing the effects of the candidate agent to the control agent. Suitable control agents include PBS or normal saline.

In another embodiment, agents such as an enzyme, or a biologically active portion thereof, which is responsible for the production or degradation of a PTK7 polypeptide or is responsible for the post-translational modification of a PTK7 polypeptide can be identified. In a primary screen, substantially pure, native or recombinantly expressed PTK7 polypeptides, nucleic acids or cellular extract or other sample comprising native or recombinantly expressed PTK7 polypeptides or nucleic acids are contacted with a plurality of candidate agents (for example but without limitation, a plurality of agents presented as a library) that may be responsible for the processing of a PTK7 polypeptide or nucleic acid, in order to identify such agents. The ability of the candidate agent to modulate the production, degradation or post-translational modification of a PTK7 polypeptide or nucleic acid can be determined by methods known to those of skill in the art, including without limitation, flow cytometry, radiolabelling, a kinase assay, a phosphatase assay, immunoprecipitation and Western blot analysis, or Northern blot analysis.

In yet another embodiment, cells expressing a PTK7 polypeptide are contacted with a plurality of candidate agents. The ability of such an agent to modulate the production, degradation or post-translational modification of a PTK7 polypeptide can be determined by methods known to those of skill in the art, as described above.

In one embodiment, agents that modulate the expression of a PTK7 polypeptide (e.g. down-regulate) are identified in a cell-based assay system. Accordingly, a population of cells expressing a PTK7 polypeptide or nucleic acid are contacted with a candidate agent and the ability of the candidate agent to alter expression of the PTK7 polypeptide or nucleic acid is determined by comparison to a reference range or control. In another embodiment, a first and second population of cells expressing a PTK7 polypeptide are contacted with a candidate agent or a control agent and the ability of the candidate agent to alter the expression of the PTK7 polypeptide or nucleic acid is determined by comparing the difference in the level of expression of the PTK7 polypeptide or nucleic acid between the first and second populations of cells. In a further embodiment, the expression of the PTK7 polypeptide or nucleic acid in the first population may be further compared to a reference range or control. If desired, this assay may be used to screen a plurality (e.g. a library) of candidate agents. The cell, for example, can be of prokaryotic origin (e.g. E. coli) or eukaryotic origin (e.g. yeast or mammalian). Further, the cells can express a PTK7 polypeptide or nucleic acid endogenously or be genetically engineered to express a PTK7 polypeptide or nucleic acid. The ability of the candidate agents to alter the expression of a PTK7 polypeptide or nucleic acid can be determined by methods known to those of skill in the art, for example and without limitation, by flow cytometry, radiolabelling, a scintillation assay, immunoprecipitation, Western blot analysis or Northern blot analysis.

In another embodiment, agents that modulate the expression of a PTK7 polypeptide or nucleic acid are identified in an animal model. Examples of suitable animals include, but are not limited to, mice, rats, rabbits, monkeys, guinea pigs, dogs and cats. Preferably, the animal used represents a model of carcinoma. Accordingly, a first and second group of mammals are administered with a candidate agent or a control agent and the ability of the candidate agent to modulate the expression of the PTK7 polypeptide or nucleic acid is determined by comparing the difference in the level of expression between the first and second group of mammals. Where desired, the expression levels of the PTK7 polypeptides or nucleic acid in the first and second groups of mammals can be compared to the level of a PTK7 polypeptide or nucleic acid in a control group of mammals. The candidate agent or a control agent can be administered by means known in the art (e.g. orally, rectally or parenterally such as intraperitoneally or intravenously). Changes in the expression of a polypeptide or nucleic acid can be assessed by the methods outlined above. In a particular embodiment, a therapeutically effective amount of an agent can be determined by monitoring an amelioration or improvement in disease symptoms, to delay onset or slow progression of the disease, for example but without limitation, a reduction in tumour size. Techniques known to physicians familiar with carcinoma can be used to determine whether a candidate agent has altered one or more symptoms associated with the disease.

One skilled in the art will also appreciate that a PTK7 polypeptide may also be used in a method for the structure-based design of an agent, in particular a small molecule which acts to modulate (e.g. stimulate or inhibit) the activity of said polypeptide, said method comprising:
1 ) determining the three-dimensional structure of said polypeptide;
2) deducing the three-dimensional structure within the polypeptide of the likely reactive or binding site(s) of the agent;
3) synthesising candidate agents that are predicted to react or bind to the deduced reactive or binding site; and
4) testing whether the candidate agent is able to modulate the activity of said polypeptide.

It will be appreciated that the method described above is likely to be an iterative process.

As discussed herein, agents which interact with a PTK7 polypeptide find use in the treatment and/or prophylaxis of carcinoma. For such use the agents will generally be administered in the form of a pharmaceutical composition. Pharmaceutical compositions may also find use as a vaccine and may comprise additional components acceptable for vaccine use and may additionally comprise one or more suitable adjuvants as known to the skilled person.

Thus, according to the invention there is provided a pharmaceutical composition comprising an agent which interacts with a PTK7 polypeptide and a pharmaceutically acceptable diluent, excipient and/or carrier.

Hereinafter, the agents of use in the invention, PTK7 polypeptides and PTK7 nucleic acids of use in treatment and/or prophylaxis are referred to as ‘active agents’. The term ‘treatment’ includes either therapeutic or prophylactic therapy. When a reference is made herein to a method of treating or preventing a disease or condition using a particular active agent or combination of agents, it is to be understood that such a reference is intended to include the use of that active agent or combination of agents in the preparation of a medicament for the treatment and/or prophylaxis of the disease or condition.

The composition will usually be supplied as part of a sterile, pharmaceutical composition that will normally include a pharmaceutically acceptable carrier. This composition may be in any suitable form (depending upon the desired method of administering it to a patient).

Active agents of the invention may be administered to a subject by any of the routes conventionally used for drug administration, for example they may be administered parenterally, orally, topically (including buccal, sublingual or transdermal) or by inhalation. The most suitable route for administration in any given case will depend on the particular active agent, the carcinoma involved, the subject, and the nature and severity of the disease and the physical condition of the subject.

The active agents may be administered in combination, e.g. simultaneously, sequentially or separately, with one or more other therapeutically active, e.g. anti-tumour, compounds.

Pharmaceutical compositions may be conveniently presented in unit dose forms containing a predetermined amount of an active agent of the invention per dose. Such a unit may contain for example but without limitation, 750 mg/kg to 0.1 mg/kg depending on the condition being treated, the route of administration and the age, weight and condition of the subject.

Pharmaceutically acceptable carriers for use in the invention may take a wide variety of forms depending, e.g. on the route of administration.

Compositions for oral administration may be liquid or solid. Oral liquid preparations may be in the form of, for example, aqueous or oily suspensions, solutions, emulsions, syrups or elixirs, or may be presented as a dry product for reconstitution with water or other suitable vehicle before use. Oral liquid preparations may contain suspending agents as known in the art.

In the case of oral solid preparations such as powders, capsules and tablets, carriers such as starches, sugars, microcrystalline cellulose, diluents, granulating agents, lubricants, binders, disintegrating agents, and the like may be included. Because of their ease of administration, tablets and capsules represent the most advantageous oral dosage unit form in which case solid pharmaceutical carriers are generally employed. In addition to the common dosage forms set out above, active agents of the invention may also be administered by controlled release means and/or delivery devices. Tablets and capsules may comprise conventional carriers or excipients such as binding agents for example, syrup, acacia, gelatin, sorbitol, tragacanth, or polyvinylpyrrolidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulphate. The tablets may be coated by standard aqueous or non-aqueous techniques according to methods well known in normal pharmaceutical practice.

Pharmaceutical compositions of the present invention suitable for oral administration may be presented as discrete units such as capsules, cachets or tablets, each containing a predetermined amount of the active agent, as a powder or granules, or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active agent with the carrier, which constitutes one or more necessary ingredients. In general, the compositions are prepared by uniformly and intimately admixing the active agent with liquid carriers or finely divided solid carriers or both, and then, if necessary, shaping the product into the desired presentation. For example, a tablet may be prepared by compression or moulding, optionally with one or more accessory ingredients.

Pharmaceutical compositions suitable for parenteral administration may be prepared as solutions or suspensions of the active agents of the invention in water suitably mixed with a surfactant such as hydroxypropylcellulose. Dispersions can also be prepared in glycerol, liquid polyethylene glycols, and mixtures thereof in oils. Under ordinary conditions of storage and use, these preparations contain a preservative to prevent the growth of microorganisms.

The pharmaceutical forms suitable for injectable use include aqueous or non-aqueous sterile injection solutions which may contain anti-oxidants, buffers, bacteriostats and solutes which render the composition isotonic with the blood of the intended recipient, and aqueous and non-aqueous sterile suspensions which may include suspending agents and thickening agents. Extemporaneous injection solutions, dispersions and suspensions may be prepared from sterile powders, granules and tablets.

Pharmaceutical compositions can be administered with medical devices known in the art. For example, in a preferred embodiment, a pharmaceutical composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the pharmaceutical compositions of the invention can be formulated to ensure proper distribution in vivo. For example, the blood-brain barrier excludes many highly hydrophilic compounds and it may be preferable to deliver pharmaceutical compositions in liposomes. Thus, in one embodiment of the invention, the active agents of the invention are formulated in liposomes; in a more preferred embodiment, the liposomes include a targeting moiety. In a most preferred embodiment, the therapeutic compounds in the liposomes are delivered by bolus injection to a site proximal to the tumour. For methods of manufacturing liposomes, see, e.g. U.S. Pat. Nos. 4,522,811; 5,374,548; and 5,399,331. The liposomes may comprise one or more moieties which are selectively transported into specific cells or organs, thus enhancing targeted drug delivery (see, e.g. Ranade, W. 1989, J. Clin. Pharmacol. 29:685). Exemplary targeting moieties include folate or biotin (see, e.g. U.S. Pat. No. 5,416,016.); mannosides (Umezawa et al., 1988, Biochem. Biophys. Res. Commun. 153:1038); antibodies (Bloeman, P G. et al., 1995, FEBS Lett. 357:140; M. Owais et al., 1995, Antimicrob. Agents Chemother. 39:180); surfactant protein A receptor (Briscoe et al., 1995, Am. J. Physiol. 1233:134), different species of which may comprise the formulations of the inventions, as well as components of the invented molecules; p120 (Schreier et al., 1994, J. Biol. Chem. 269:9090); see also Keinanen, K. & Laukkanen, M L. 1994, FEBS Lett. 346:123; Killion, J J. & Fidler, I J. 1994, Immunomethods 4:273. The compositions may be presented in unit-dose or multi-dose containers, for example in sealed ampoules and vials and to enhance stability, may be stored in a freeze-dried (lyophilized) condition requiring only the addition of the sterile liquid carrier, for example water for injections, immediately prior to use. The sterile liquid carrier may be supplied in a separate vial or ampoule and can be a solvent or dispersion medium containing, for example, water, ethanol, polyol (e.g. glycerol, propylene glycol and liquid polyethylene glycol), suitable mixtures thereof, and vegetable oils. Advantageously, agents such as a local anaesthetic, preservative and buffering agents can be included the sterile liquid carrier.

Pharmaceutical compositions adapted for topical administration may be formulated as ointments, creams, suspensions, lotions, powders, solutions, pastes, gels, impregnated dressings, sprays, aerosols or oils, transdermal devices, dusting powders, and the like. These compositions may be prepared via conventional methods containing the active agent. Thus, they may also comprise compatible conventional carriers and additives, such as preservatives, solvents to assist drug penetration, emollients in creams or ointments and ethanol or oleyl alcohol for lotions. Such carriers may be present as from about 1% up to about 98% of the composition. More usually they will form up to about 80% of the composition. As an illustration only, a cream or ointment is prepared by mixing sufficient quantities of hydrophilic material and water, containing from about 5-10% by weight of the compound, in sufficient quantities to produce a cream or ointment having the desired consistency.

Pharmaceutical compositions adapted for transdermal administration may be presented as discrete patches intended to remain in intimate contact with the epidermis of the recipient for a prolonged period of time. For example, the active agent may be delivered from the patch by iontophoresis.

For applications to external tissues, for example the mouth and skin, the compositions are preferably applied as a topical ointment or cream. When formulated in an ointment, the active agent may be employed with either a paraffinic or a water-miscible ointment base. Alternatively, the active agent may be formulated in a cream with an oil-in-water cream base or a water-in-oil base.

Pharmaceutical compositions adapted for topical administration in the mouth include lozenges, pastilles and mouth washes.

Pharmaceutical compositions adapted for topical administration to the eye include eye drops wherein the active agent is dissolved or suspended in a suitable carrier, especially an aqueous solvent. They also include topical ointments or creams as above.

Pharmaceutical compositions suitable for rectal administration wherein the carrier is a solid are most preferably presented as unit dose suppositories. Suitable carriers include cocoa butter or other glyceride or materials commonly used in the art, and the suppositories may be conveniently formed by admixture of the combination with the softened or melted carrier(s) followed by chilling and shaping moulds. They may also be administered as enemas.

Pharmaceutical compositions adapted for vaginal administration may be presented as pessaries, tampons, creams, gels, pastes, foams or spray compositions. These may comprise emollients or bases as commonly used in the art.

The dosage to be administered of an active agent will vary according to the particular active agent, the carcinoma involved, the subject, and the nature and severity of the disease and the physical condition of the subject, and the selected route of administration; the appropriate dosage can be readily determined by a person skilled in the art. For the treatment and/or prophylaxis of carcinoma in humans and animals pharmaceutical compositions comprising antibodies can be administered to patients (e.g., human subjects) at therapeutically or prophylactically effective dosages (e.g. dosages which result in tumour growth inhibition and/or tumour cell migration inhibition) using any suitable route of administration, such as injection and other routes of administration known in the art for antibody-based clinical products.

The compositions may contain from 0.1% by weight, preferably from 10-60%, or more, by weight, of the active agent of the invention, depending on the method of administration.

It will be recognized by one of skill in the art that the optimal quantity and spacing of individual dosages of an active agent of the invention will be determined by the nature and extent of the condition being treated, the form, route and site of administration, and the age and condition of the particular subject being treated, and that a physician will ultimately determine appropriate dosages to be used. This dosage may be repeated as often as appropriate. If side effects develop the amount and/or frequency of the dosage can be altered or reduced, in accordance with normal clinical practice.

PTK7 polypeptides may also be of use in the treatment and/or prophylaxis of carcinoma, e.g. when administered as a vaccine. Where they are provided for use with the methods of the invention they are preferably provided in isolated form. More preferably the PTK7 polypeptides have been purified to at least some extent. PTK7 polypeptides can also be produced using recombinant methods, synthetically produced or produced by a combination of these methods. PTK7 polypeptides may be provided in substantially pure form, that is to say free, to a substantial extent, from other proteins.

Recombinant PTK7 polypeptides may be prepared by processes well known in the art from genetically engineered host cells comprising expression systems. Accordingly, the present invention also relates to expression systems which comprise a PTK7 polypeptide or PTK7 nucleic acid, to host cells which are genetically engineered with such expression systems and to the production of PTK7 polypeptides by recombinant techniques. Cell-free translation systems systems can also be employed to produce recombinant polypeptides (e.g. rabbit reticulocyte lysate, wheat germ lysate, SP6/T7 in vitro T&T and RTS 100 E. Coli HY transcription and translation kits from Roche Diagnostics Ltd., Lewes, UK and the TNT Quick coupled Transcription/Translation System from Promega UK, Southampton, UK.

For recombinant PTK7 polypeptide production, host cells can be genetically engineered to incorporate expression systems or portions thereof for PTK7 nucleic acids. Such incorporation can be performed using methods well known in the art, such as, calcium phosphate transfection, DEAD-dextran mediated transfection, transvection, microinjection, cationic lipid-mediated transfection, electroporation, transduction, scrape loading, ballistic introduction or infection (see e.g. Davis et al., Basic Methods in Molecular Biology, 1986 and Sambrook et al., Molecular Cloning: A Laboratory Manual, 2^{nd} Ed., Cold Spring Harbour laboratory Press, Cold Spring Harbour, N.Y., 1989).

Representative examples of host cells include bacterial cells e.g. E. Coli, Streptococci, Staphylococci, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, HEK 293, BHK and Bowes melanoma cells; and plant cells.

A wide variety of expression systems can be used, such as and without limitation, chromosomal, episomal and virus-derived systems, e.g. vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector which is able to maintain, propagate or express a nucleic acid to produce a polypeptide in a host may be used. The appropriate nucleic acid sequence may be inserted into an expression system by any variety of well-known and routine techniques, such as those set forth in Sambrook et al., supra. Appropriate secretion signals may be incorporated into the PTK7 polypeptide to allow secretion of the translated protein into the lumen of the endoplasmic reticulum, the periplasmic space or the extracellular environment. These signals may be endogenous to the PTK7 polypeptide or they may be heterologous signals.

If a PTK7 polypeptide is to be expressed for use in cell-based screening assays, it is preferred that the polypeptide be produced at the cell surface. In this event, the cells may be harvested prior to use in the screening assay. If the PTK7 polypeptide is secreted into the medium, the medium can be recovered in order to isolate said polypeptide. If produced intracellularly, the cells must first be lysed before the PTK7 polypeptide is recovered.

PTK7 polypeptides can be recovered and purified from recombinant cell cultures or from other biological sources by well-known methods including, ammonium sulphate or ethanol precipitation, acid extraction, anion or cation exchange chromatography, phosphocellulose chromatography, affinity chromatography, hydrophobic interaction chromatography, hydroxylapatite chromatography, molecular sieving chromatography, centrifugation methods, electrophoresis methods and lectin chromatography. In one embodiment, a combination of these methods is used. In another embodiment, high performance liquid chromatography is used. In a further embodiment, an antibody which specifically binds to a PTK7 polypeptide can be used to deplete a sample comprising a PTK7 polypeptide of said polypeptide or to purify said polypeptide. Techniques well-known in the art, may be used for refolding to regenerate native or active conformations of the PTK7 polypeptides when the polypeptides have been denatured during isolation and or purification. In the context of the present invention, PTK7 polypeptides can be obtained from a biological sample from any source, such as and without limitation, breast, kidney, pancreas, bladder, ovary or lung tissue.

PTK7 polypeptides may be in the form of a ‘mature’ protein or may be part of a larger protein such as a fusion protein. It is often advantageous to include an additional amino acid sequence which contains secretory or leader sequences, a pre-, pro- or prepro-protein sequence, or a sequence which aids in purification such as an affinity tag, for example, but without limitation, multiple histidine residues, a FLAG tag, HA tag or myc tag. An additional sequence which may provide stability during recombinant production may also be used. Such sequences may be optionally removed as required by incorporating a cleavable sequence as an additional sequence or part thereof. Thus, a PTK7 polypeptide may be fused to other moieties including other polypeptides. Such additional sequences and affinity tags are well known in the art.

Amino acid substitutions may be conservative or semi-conservative as known in the art and preferably do not significantly affect the desired activity of the polypeptide. Substitutions may be naturally occurring or may be introduced for example using mutagenesis (e.g. Hutchinson et al., 1978, J. Biol. Chem. 253:6551). Thus, the amino acids glycine, alanine, valine, leucine and isoleucine can often be substituted for one another (amino acids having aliphatic side chains). Of these possible substitutions, it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains);
cysteine and methionine (amino acids having sulphur-containing side chains); and
aspartic acid and glutamic acid can substitute for phospho-serine and phospho-threonine, respectively (amino acids with acidic side chains).

In one particular embodiment, the substituted amino acid(s) do significantly affect the activity of the PTK7 polypeptide and may be selected specifically to render dominant negative activity upon the peptide. In another embodiment, the substituted amino acid(s) may be selected specifically to render the polypeptide constitutively active.

Modifications include naturally occurring modifications such as and without limitation, post-translational modifications and also non-naturally occurring modifications such as may be introduced by mutagenesis.

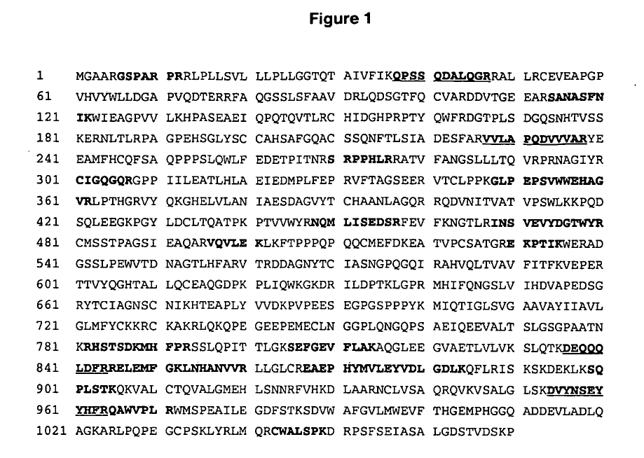

Preferably a derivative of a PTK7 polypeptide has at least 70% identity to the amino acid sequence shown in FIG. 1 (SEQ ID NO: 1), more preferably it has at least 75%, at least 80%, at least 85%, at least 90%, at least 95% or at least 98% identity. Percentage identity is a well known concept in the art and can be calculated using, for example but without limitation, the BLAST™ software available from NCBI (Altschul, S. F. et al., 1990, J. Mol. Biol. 215:403-410; Gish, W. & States, D. J. 1993, Nature Genet. 3:266-272. Madden, T. L. et al., 1996, Meth. Enzymol. 266:131-141; Altschul, S. F. et al., 1997, Nucleic Acids Res. 25:3389-3402); Zhang, J. & Madden, T. L. 1997, Genome Res. 7:649-656).

A fragment of a PTK7 polypeptide may also be of use in the methods of the invention and includes a fragment of a polypeptide having the amino acid sequence of SEQ ID NO:1, which has at least 70% homology over the length of the fragment. Preferably, said fragments are at least 10 amino acids in length, preferably they are at least 20, at least 30, at least 50 or at least 100 amino acids in length. A fragment has at least 70% identity over its length to the amino acid sequence shown in FIG. 1 (SEQ ID NO: 1), more preferably it has at least 75%, at least 80%, at least 85%, at least 90%, at least 95% or at least 98% identity.

Where a PTK7 polypeptide is the active agent of a pharmaceutical composition for use in the treatment and/or prophylaxis of carcinoma, preferably recombinant PTK7 polypeptides are used. In a particular embodiment, a PTK7 polypeptide is fused to another polypeptide, such as the protein transduction domain of the HIV/Tat protein, which facilitates the entry of the fusion protein into a cell (Asoh, S. et al., 2002, Proc. Natl. Acad. Sci. USA, 99:17107-17112) is provided for use in the manufacture of a medicament for the treatment and/or prophylaxis of carcinoma.

In another aspect, detection of a PTK7 polypeptide in a subject with carcinoma may be used to identify in particular an appropriate patient population for treatment according to the methods of the invention.

Accordingly, the present invention provides a method of screening for and/or diagnosis or prognosis of carcinoma in a subject, and/or monitoring the effectiveness of carcinoma therapy, which comprises the step of detecting and/or quantifying in a biological sample obtained from said subject, a PTK7 polypeptide. The PTK7 polypeptide for use in the method of screening and/or diagnosis preferably:
(a) comprises or consists of the amino acid sequence of SEQ ID NO:1;
(b) is a derivative having one or more amino acid substitutions, modifications, deletions or insertions relative to the amino acid sequence of SEQ ID NO:1 which retains the activity of PTK7; or
(c) is a fragment of a polypeptide having the amino acid sequence of SEQ ID NO:1, which is at least ten amino acids long and has at least 70% homology over the length of the fragment.

In one aspect, the expression is compared to a previously determined reference range.

Preferably, the step of detecting comprises:
(a) contacting the sample with a capture reagent that is specific for a polypeptide as defined in (a) to (c), above; and
(b) detecting whether binding has occurred between the capture reagent and said polypeptide in the sample.

In another aspect, the captured polypeptide is detected using a directly or indirectly labelled detection reagent which may be immobilised on a solid phase.

In the context of the present invention a biological sample may obtained from any source, for example but without limitation, a tissue biopsy.

A convenient means for detecting/quantifying a PTK7 polypeptide involves the use of antibodies. A PTK7 polypeptide can be used as an immunogen to raise antibodies which interact with (bind to or recognise) said polypeptide using methods known in the art as described above. Thus, in a further aspect, the present invention provides the use of an antibody that specifically binds to at least one PTK7 polypeptide for screening for and/or diagnosis of carcinoma in a subject or for monitoring the efficacy of an anti-carcinoma therapy. In a particular embodiment, the methods of diagnosis using an anti-PTK7 polypeptide antibody can be used to identify an appropriate patient population for treatment according to the methods of the invention.

PTK7 antibodies can also be used, inter alia, for the diagnosis of carcinoma by detecting PTK7 expression in a biological sample of human tissue and/or in subfractions thereof, for example but without limitation, membrane, cytosolic or nuclear subfractions.

In a further aspect, the method of detecting a PTK7 polypeptide in a biological sample comprises detecting and/or quantitating the amount of the PTK7 polypeptide in said sample using a directly or indirectly labelled detection reagent. A PTK7 polypeptide can be detected by means of any immunoassay known in the art, including, without limitation, immunoprecipitation followed by sodium dodecyl sulfate polyacrylamide gel electrophoresis, 2 dimensional gel electrophoresis, competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays.

Detection of the interaction of an antibody with an antigen can be facilitated by coupling the antibody to a detectable substance for example, but without limitation, an enzyme (such as horseradish peroxidase, alkaline phosphatase, beta-galactosidase, acetylcholinesterase), a prosthetic group (such as streptavidin, avidin, biotin), a fluorescent material (such as umbelliferone, fluorescein, fluorescein isothiocyanate, rhodamine, dichlorotriazinylamine fluorescein, dansyl chloride, phycoerythrin), a luminescent material (such as luminol), a bioluminescent material (such as luciferase, luciferin, aequorin), a radioactive nuclide (such as ¹²⁵I, ¹³¹I, ¹¹¹In, ⁹⁹Tc) a positron emitting metal or a non-radioactive paramagnetic metal ion (see U.S. Pat. No. 4,741,900).

The invention also provides diagnostic kits, comprising a capture reagent (e.g. an antibody) against a PTK7 polypeptide as defined above. In addition, such a kit may optionally comprise one or more of the following:

(1) instructions for using the capture reagent for screening, diagnosis, prognosis, therapeutic monitoring or any combination of these applications;

(2) a labelled binding partner to the capture reagent;

(3) a solid phase (such as a reagent strip) upon which the capture reagent is immobilised; and

(4) a label or insert indicating regulatory approval for screening, diagnostic, prognostic or therapeutic use or any combination thereof.

If no labelled binding partner to the capture reagent is provided, the anti-PTK7 polypeptide capture reagent itself can be labelled with a detectable marker, e.g. a chemiluminescent, enzymatic, fluorescent, or radioactive moiety (see above).

It will be apparent to one skilled in the art that detection and/or quantitation of a PTK7 nucleic acid may also be used in a method of screening for and/or diagnosis or prognosis of carcinoma in a subject, and/or monitoring the effectiveness of carcinoma therapy.

Unless the context indicates otherwise, PTK7 nucleic acids include those nucleic acid molecules which may have one or more of the following characteristics and thus may:
d) comprise or consist of the DNA sequence of SEQ ID NO:2 or its RNA equivalent;
e) have a sequence which is complementary to the sequences of d);
f) have a sequence which codes for a PTK7 polypeptide;
g) have a sequence which shows substantial identity with any of those of d), e) and f); or
h) is a fragment of d), e), f) or g), which is at least 10 nucleotides in length; and may have one or more of the following characteristics:
1) they may be DNA or RNA;
2) they may be single or double stranded;
3) they may be in substantially pure form. Thus, they may be provided in a form which is substantially free from contaminating proteins and/or from other nucleic acids; and
4) they may be with introns or without introns (e.g. as cDNA).

Fragments of PTK7 nucleic acids are preferably at least 20, at least 30, at least 50, at least 100 or at least 250 nucleotides in length.

The invention also provides the use of nucleic acids which are complementary to the PTK7 nucleic acids described in (d)-(h) above, and can hybridise to said PTK7 nucleic acids. Such nucleic acid molecules are referred to as “hybridising” nucleic acid molecules. For example, but without limitation, hybridising nucleic acid molecules can be useful as probes or primers. Hybridising nucleic acid molecules may have a high degree of sequence identity along its length with a nucleic acid molecule within the scope of (d)-(h) above (e.g. at least 50%, at least 75%, at least 80%, at least 85%, at least 90%, at least 95%, or at least 98% sequence identity). The use of hybridising nucleic acid molecules that can hybridise to any of the nucleic acid molecules discussed above, e.g. in hybridising assays, is also covered by the present invention.

Hybridisation assays can be used for screening, prognosis, diagnosis, or monitoring of therapy of carcinoma in a subject. Accordingly, such a hybridisation assay comprises:

i) contacting a biological sample, obtained from a subject, containing nucleic acid with a nucleic acid probe capable of hybridising to a PTK7 nucleic acid molecule, under conditions such that hybridisation can occur; and

ii) detecting or measuring any resulting hybridisation.

Preferably, such hybridising molecules are at least 10 nucleotides in length and are preferably at least 25 or at least 50 nucleotides in length. More preferably, the hybridising nucleic acid molecules specifically hybridise to nucleic acids within the scope of any one of (d) to (f), above. Most preferably, the hybridisation occurs under stringent hybridisation conditions. One example of stringent hybridisation conditions is where attempted hybridisation is carried out at a temperature of from about 35° C. to about 65° C. using a salt solution which is about 0.9M. However, the skilled person will be able to vary such conditions as appropriate in order to take into account variables such as probe length, base composition, type of ions present, etc.

The invention also provides a diagnostic kit comprising a nucleic acid probe capable of hybridising to RNA encoding a PTK7 polypeptide, suitable reagents and instructions for use.

In a further embodiment, a diagnostic kit is provided comprising in one or more containers a pair of primers that under appropriate reaction conditions can prime amplification of at least a portion of a PTK7 nucleic acid molecule, such as by polymerase chain reaction (see e.g. Innis et al., 1990, PCR Protocols, Academic Press, Inc., San Diego, Calif.), ligase chain reaction (see EP 320,308) use of Qβ replicase, cyclic probe reaction, or other methods known in the art. Typically, primers are at least eight nucleotides long and will preferably be at least ten to twenty-five nucleotides long and more preferably fifteen to twenty-five nucleotides long. In some cases, primers of at least thirty or at least thirty-five nucleotides in length may be used.

In yet another aspect, the present invention provides the use of at least one PTK7 nucleic acid in the manufacture of a medicament for use in the treatment and/or prophylaxis of carcinoma.

In a specific embodiment, hybridising PTK7 nucleic acid molecules are used as anti-sense molecules, to alter the expression of PTK7 polypeptides by binding to complementary PTK7 nucleic acids and can be used in the treatment and/or prophylaxis of carcinoma. An antisense nucleic acid includes a PTK7 nucleic acid capable of hybridising by virtue of some sequence complementarity to a portion of an RNA (preferably mRNA) encoding a PTK7 polypeptide. The antisense nucleic acid can be complementary to a coding and/or non-coding region of an mRNA encoding such a polypeptide. Most preferably, expression of a PTK7 polypeptide is inhibited by use of antisense nucleic acids. Thus, the present invention provides the therapeutic or prophylactic use of nucleic acids comprising at least eight nucleotides that are antisense to a gene or cDNA encoding a PTK7 polypeptide.

In another embodiment, symptoms of carcinoma may be ameliorated by decreasing the level or activity of a PTK7 polypeptide by using gene sequences encoding a polypeptide as defined herein in conjunction with well-known gene “knock-out,” ribozyme or triple helix methods to decrease gene expression of the polypeptide. In this approach, ribozyme or triple helix molecules are used to modulate the activity, expression or synthesis of the gene, and thus to ameliorate the symptoms of carcinoma. Such molecules may be designed to reduce or inhibit expression of a mutant or non-mutant target gene. Techniques for the production and use of such molecules are well known to those of skill in the art.

Endogenous PTK7 polypeptide expression can also be reduced by inactivating or “knocking out” the gene encoding the polypeptide, or the promoter of such a gene, using targeted homologous recombination (e.g. see Smithies, et al., 1985, Nature 317:230-234; Thomas & Capecchi, 1987, Cell 51:503-512; Thompson et al., 1989, Cell 5:313-321; and Zijlstra et al., 1989, Nature 342:435-438). For example, a mutant gene encoding a non-functional polypeptide (or a completely unrelated DNA sequence) flanked by DNA homologous to the endogenous PTK7 gene (either the coding regions or regulatory regions of the gene encoding the polypeptide) can be used, with or without a selectable marker and/or a negative selectable marker, to transfect cells that express the target gene in vivo. Insertion of the DNA construct, via targeted homologous recombination, results in inactivation of the target gene.

In another embodiment, the nucleic acid is administered via gene therapy (see for example Hoshida, T. et al., 2002, Pancreas, 25:111-121; Ikuno, Y. 2002, Invest. Ophthalmol. Vis. Sci. 2002 43:2406-2411; Bollard, C., 2002, Blood 99:3179-3187; Lee E., 2001, Mol. Med. 7:773-782). Gene therapy refers to administration to a subject of an expressed or expressible PTK7 nucleic acid. Any of the methods for gene therapy available in the art can be used according to the present invention.

Delivery of the therapeutic PTK7 nucleic acid into a patient can be direct in vivo gene therapy (i.e. the patient is directly exposed to the nucleic acid or nucleic acid-containing vector) or indirect ex vivo gene therapy (i.e. cells are first transformed with the nucleic acid in vitro and then transplanted into the patient).

For example for in vivo gene therapy, an expression vector containing the PTK7 nucleic acid is administered in such a manner that it becomes intracellular, i.e. by infection using a defective or attenuated retroviral or other viral vectors as described, for example, in U.S. Pat. No. 4,980,286 or by Robbins et al., 1998, Pharmacol. Ther. 80:35-47.

The various retroviral vectors that are known in the art are such as those described in Miller et al. (1993, Meth. Enzymol. 217:581-599) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek (1999, Human Gene Therapy, 10:2451-2459). Chimeric viral vectors that can be used are those described by Reynolds et al. (1999, Molecular Medicine Today, 1:25-31). Hybrid vectors can also be used and are described by Jacoby et al. (1997, Gene Therapy, 4:1282-1283).

Direct injection of naked DNA or through the use of microparticle bombardment (e.g. Gene Gun®; Biolistic, Dupont) or by coating it with lipids can also be used in gene therapy. Cell-surface receptors/transfecting compounds or through encapsulation in liposomes, microparticles or microcapsules or by administering the nucleic acid in linkage to a peptide which is known to enter the nucleus or by administering it in linkage to a ligand predisposed to receptor-mediated endocytosis (See Wu & Wu, 1987, J. Biol. Chem., 262:4429-4432) can be used to target cell types which specifically express the receptors of interest.

In another embodiment a nucleic acid ligand compound comprising a PTK7 nucleic acid can be produced in which the ligand comprises a fusogenic viral peptide designed so as to disrupt endosomes, thus allowing the PTK7 nucleic acid to avoid subsequent lysosomal degradation. The PTK7 nucleic acid can be targeted in vivo for cell specific endocytosis and expression by targeting a specific receptor such as that described in WO 92/06180, WO93/14188 and WO 93/20221. Alternatively the nucleic acid can be introduced intracellularly and incorporated within the host cell genome for expression by homologous recombination (See Zijlstra et al, 1989, Nature, 342:435-428).

In ex vivo gene therapy, a gene is transferred into cells in vitro using tissue culture and the cells are delivered to the patient by various methods such as injecting subcutaneously, application of the cells into a skin graft and the intravenous injection of recombinant blood cells such as haematopoietic stem or progenitor cells.

Cells into which a PTK7 nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryotcytes, granulocytes, haematopoietic cells or progenitor cells, and the like.

In a one aspect, the pharmaceutical composition comprises a PTK7 nucleic acid, said nucleic acid being part of an expression vector that expresses a PTK7 polypeptide or chimeric protein thereof in a suitable host. In particular, such a nucleic acid has a promoter operably linked to the polypeptide coding region, said promoter being inducible or constitutive (and, optionally, tissue-specific). In another particular embodiment, a nucleic acid molecule is used in which the coding sequences and any other desired sequences are flanked by regions that promote homologous recombination at a desired site in the genome, thus providing for intrachromosomal expression of the nucleic acid (Koller & Smithies, 1989, Proc. Natl. Acad. Sci. USA 86:8932-8935; Zijlstra et al., 1989, Nature 342:435-438).

PTK7 nucleic acids may be obtained using standard cloning and screening techniques, from a cDNA library derived from mRNA in human cells, using expressed sequence tag (EST) analysis (Adams, M. et al., 1991, Science, 252:1651-1656; Adams, M. et al., 1992, Nature 355:632-634; Adams, M. et al., 1995, Nature, 377:Suppl: 3-174). PTK7 nucleic acids can also be obtained from natural sources such as genomic DNA libraries or can be synthesized using well known and commercially available techniques. The PTK7 nucleic acids comprising coding sequence for PTK7 polypeptides described above can be used for the recombinant production of said polypeptides. The PTK7 nucleic acids may include the coding sequence for the mature polypeptide, by itself; or the coding sequence for the mature polypeptide in reading frame with other coding sequences, such as those encoding a leader or secretory sequence, a pre-, pro- or prepro-protein sequence, a cleavable sequence or other fusion peptide portions, such as an affinity tag or an additional sequence conferring stability during production of the polypeptide. Preferred affinity tags include multiple histidine residues (for example see Gentz et al., 1989, Proc. Natl. Acad. Sci USA 86:821-824), a FLAG tag, HA tag or myc tag. The PTK7 nucleic acids may also contain non-coding 5′ and 3′ sequences, such as transcribed, non-translated sequences, splicing and polyadenylation signals, ribosome binding sites and sequences that stabilize mRNA.

PTK7 polypeptide derivatives can be created by introducing one or more nucleotide substitutions, additions or deletions into the nucleotide sequence of a PTK7 nucleic acid such that one or more amino acid substitutions, additions or deletions are introduced into the encoded protein. Standard techniques known to those of skill in the art can be used to introduce mutations, including, for example, site-directed mutagenesis and PCR-mediated mutagenesis. Preferably, conservative amino acid substitutions are made at one or more predicted non-essential amino acid residues.

A PTK7 nucleic acid encoding a PTK7 polypeptide, including homologues and orthologues from species other than human, may be obtained by a process which comprises the steps of screening an appropriate library under stringent hybridisation conditions with a labelled probe having the sequence of a PTK7 nucleic acid as described in (d)-(h) above, and isolating full-length cDNA and genomic clones containing said nucleic acid sequence. Such hybridisation techniques are well-known in the art. One example of stringent hybridisation conditions is where attempted hybridisation is carried out at a temperature of from about 35° C. to about 65° C. using a salt solution of about 0.9M. However, the skilled person will be able to vary such conditions as appropriate in order to take into account variables such as probe length, base composition, type of ions present, etc. For a high degree of selectivity, relatively stringent conditions such as low salt or high temperature conditions, are used to form the duplexes. Highly stringent conditions include hybridisation to filter-bound DNA in 0.5M NaHPO₄, 7% sodium dodecyl sulphate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3). For some applications, less stringent conditions for duplex formation are required. Moderately stringent conditions include washing in 0.2×SSC/0.1% SDS at 42° C. (Ausubel et al., 1989, supra). Hybridisation conditions can also be rendered more stringent by the addition of increasing amounts of formamide, to destabilise the hybrid duplex. Thus, particular hybridisation conditions can be readily manipulated, and will generally be chosen as appropriate. In general, convenient hybridisation temperatures in the presence of 50% formamide are: 42° C. for a probe which is 95-100% identical to the fragment of a gene encoding a polypeptide as defined herein, 37° C. for 90-95% identity and 32° C. for 70-90% identity.

One skilled in the art will understand that, in many cases, an isolated cDNA sequence will be incomplete, in that the region coding for the polypeptide is cut short at the 5′ end of the cDNA. This is a consequence of reverse transcriptase, an enzyme with inherently low processivity (a measure of the ability of the enzyme to remain attached to the template during the polymerization reaction), failing to complete a DNA copy of the mRNA template during 1 ^{st} strand cDNA synthesis.

Methods to obtain full length cDNAs or to extend short cDNAs are well known in the art, for example RACE (Rapid amplification of cDNA ends; e.g. Frohman et al., 1988, Proc. Natl. Acad. Sci USA 85:8998-9002). Recent modifications of the technique, exemplified by the Marathon™ technology (Clontech Laboratories Inc.) have significantly simplified the search for longer cDNAs. This technology uses cDNAs prepared from mRNA extracted from a chosen tissue followed by the ligation of an adaptor sequence onto each end. PCR is then carried out to amplify the missing 5′-end of the cDNA using a combination of gene specific and adaptor specific oligonucleotide primers. The PCR reaction is then repeated using nested primers which have been designed to anneal with the amplified product, typically an adaptor specific primer that anneals further 3′ in the adaptor sequence and a gene specific primer that anneals further 5′ in the known gene sequence. The products of this reaction can then be analysed by DNA sequencing and a full length cDNA constructed either by joining the product directly to the existing cDNA to give a complete sequence, or carrying out a separate full length PCR using the new sequence information for the design of the 5′ primer.

A further aspect of the invention relates to a vaccine composition of use in the treatment and/or prophylaxis of carcinoma. A PTK7 polypeptide or nucleic acid as described above can be used in the production of vaccines for treatment and/or prophylaxis of carcinoma. Such material can be antigenic and/or immunogenic. Antigenic includes a protein or nucleic acid that is capable of being used to raise antibodies or indeed is capable of inducing an antibody response in a subject. Immunogenic material includes a protein or nucleic acid that is capable of eliciting an immune response in a subject. Thus, in the latter case, the protein or nucleic acid may be capable of not only generating an antibody response but, in addition, a non-antibody based immune responses, i.e. a cellular or humoral response. It is well known in the art that is possible to identify those regions of an antigenic or immunogenic polypeptide that are responsible for the antigenicity or immunogenicity of said polypeptide, i.e. an epitope or epitopes. Amino acid and peptide characteristics well known to the skilled person can be used to predict the antigenic index (a measure of the probability that a region is antigenic) of a PTK7 polypeptide. For example, but without limitation, the ‘Peptidestructure’ program (Jameson and Wolf, 1988, CABIOS, 4(1):181) and a technique referred to as ‘Threading’ (Altuvia Y. et al., 1995, J. Mol. Biol. 249:244) can be used. Thus, the PTK7 polypeptides may include one or more such epitopes or be sufficiently similar to such regions so as to retain their antigenic/immunogenic properties.

Since a polypeptide or a nucleic acid may be broken down in the stomach, the vaccine composition is preferably administered parenterally (e.g. subcutaneous, intramuscular, intravenous or intradermal injection).

Accordingly, in further embodiments, the present invention provides:
a) the use of such a vaccine in inducing an immune response in a subject; and
b) a method for the treatment and/or prophylaxis of carcinoma in a subject, or of vaccinating a subject against carcinoma which comprises the step of administering to the subject an effective amount of a PTK7 polypeptide or nucleic acid, preferably as a vaccine.

Preferred features of each embodiment of the invention are as for each of the other embodiments mutatis mutandis. All publications, including but not limited to patents and patent applications cited in this specification are herein incorporated by reference as if each individual publication were specifically and individually indicated to be incorporated by reference herein as though fully set forth.

The invention will now be described with reference to the following examples, which are merely illustrative and should not in any way be construed as limiting the scope of the present invention.

FIG. 1 shows the protein sequence of PTK7 (AAC50484/JC4593), SEQ ID NO:1. The tandem mass spectrum peptides are in bold and underlined, MALDI mass spectra peptides are in bold.

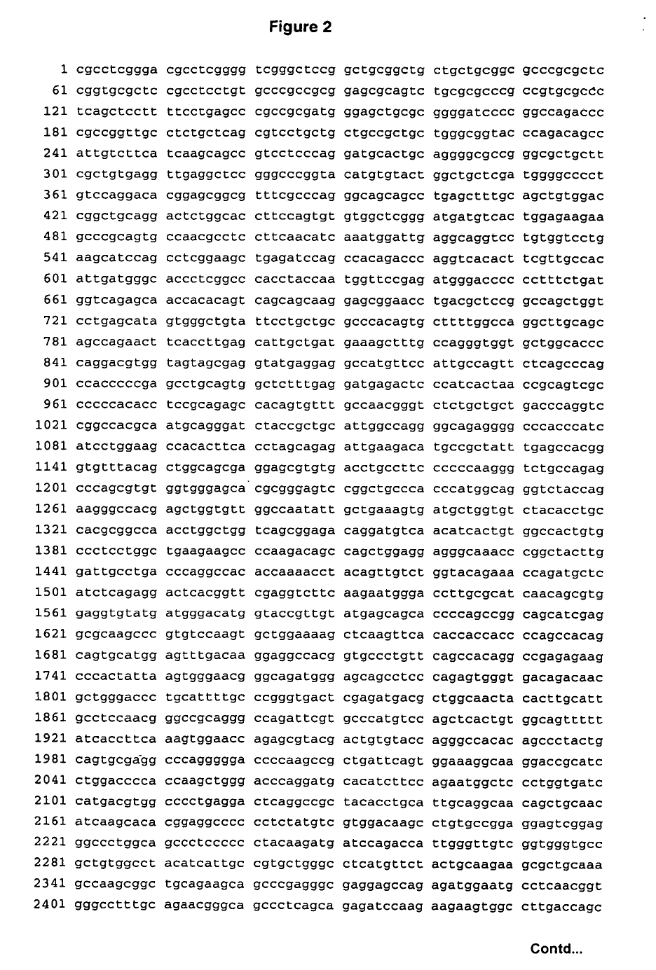

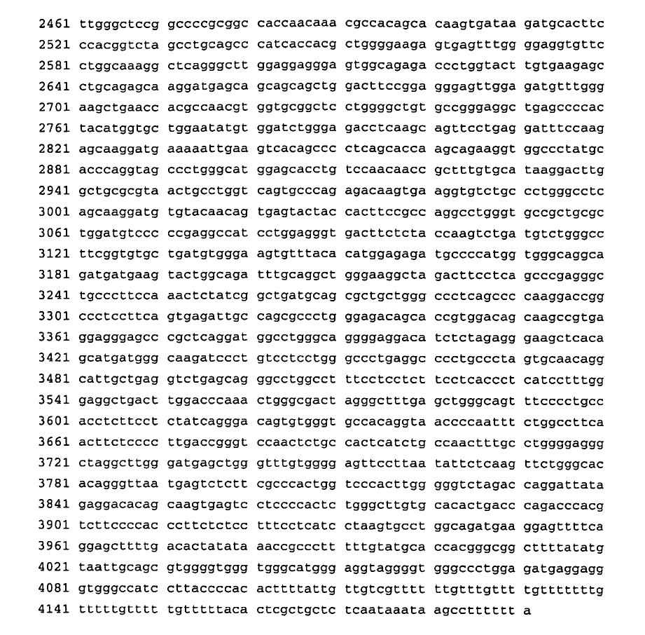

FIG. 2 shows the nucleic acid sequence of PTK7 (U40271), SEQ ID NO:2.

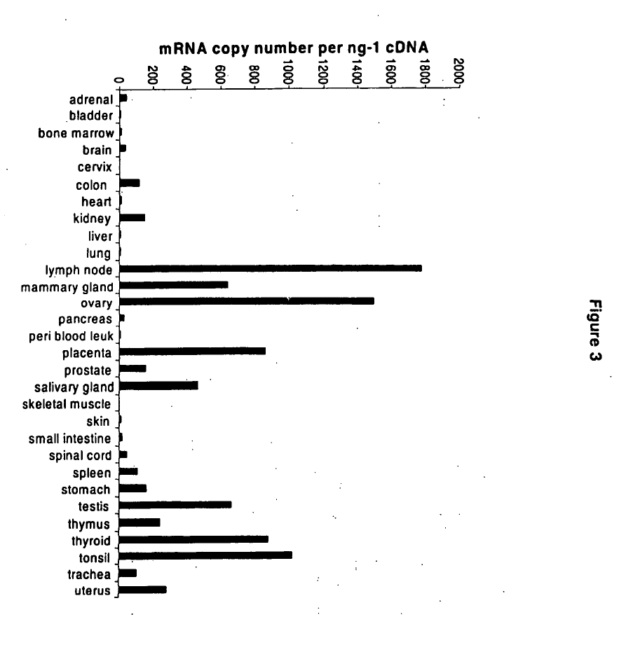

FIG. 3 shows the distribution of PTK7 mRNA in normal tissues; mRNA levels were quantified by real time RT-PCR and are expressed as the number of copies ng⁻¹ cDNA.

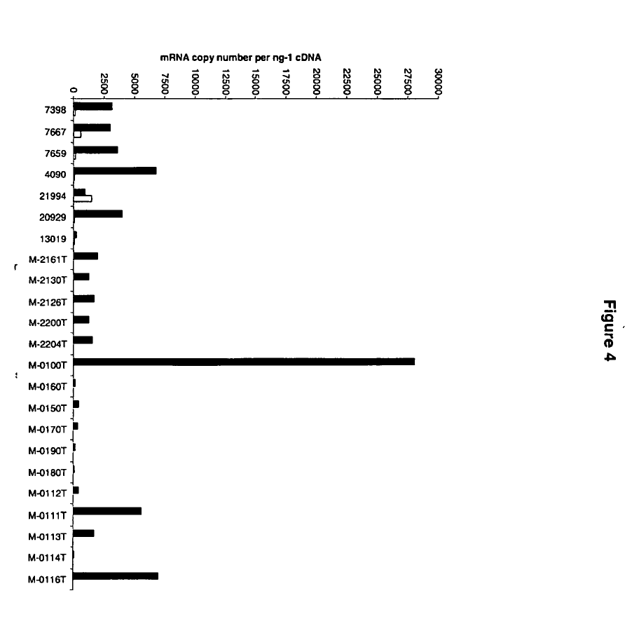

FIG. 4 shows the distribution of PTK7 mRNA in patient matched adjacent normal (open bars) and tumour (black bars) breast tissues; mRNA levels were quantified by real time RT-PCR and are expressed as the number of copies ng⁻¹ cDNA.

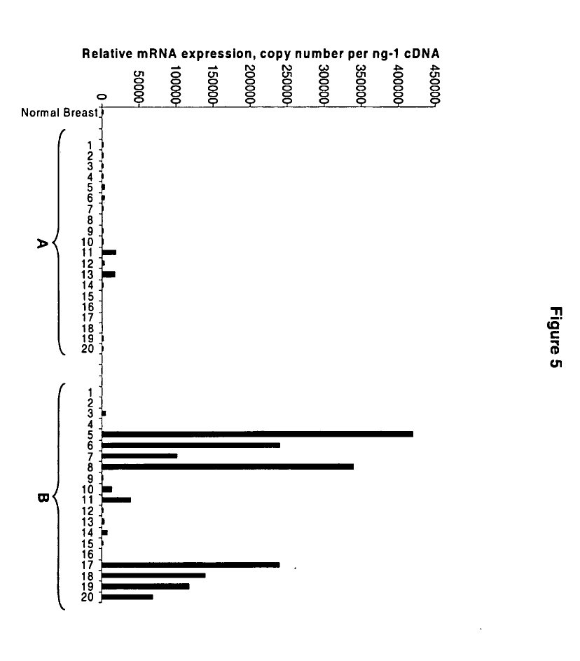

FIG. 5 shows the distribution of PTK7 mRNA in breast tumour tissues. 40 tumour samples were obtained from patients without (A) or with (B) lymph node metastasis; mRNA levels were quantified by real time RT-PCR and are expressed as the number of copies ng⁻¹ cDNA.

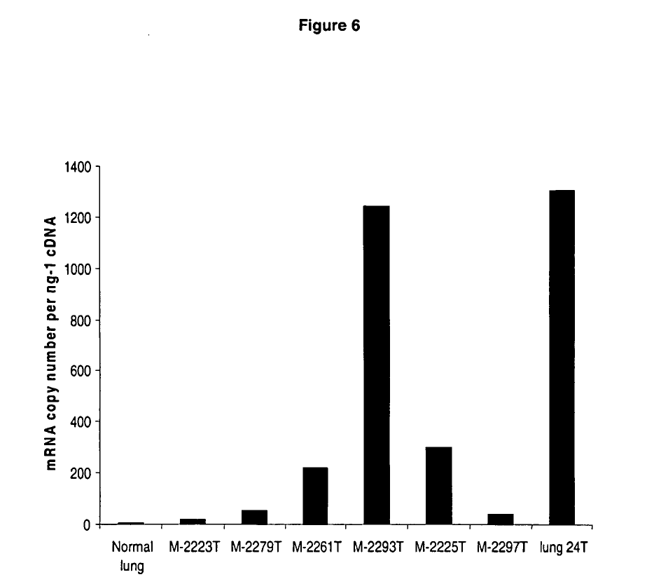

FIG. 6 shows the distribution of PTK7 mRNA in matched normal and tumour lung tissues; mRNA levels were quantified by real time RT-PCR and are expressed as the number of copies ng⁻¹ cDNA.

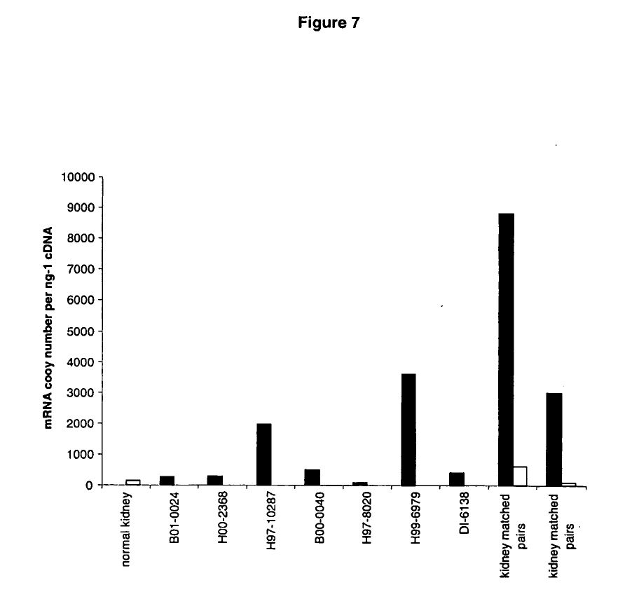

FIG. 7 shows the distribution of PTK7 mRNA in matched normal (open bars) and tumour kidney tissues (black bars); mRNA levels were quantified by real time RT-PCR and are expressed as the number of copies ng⁻¹ cDNA.

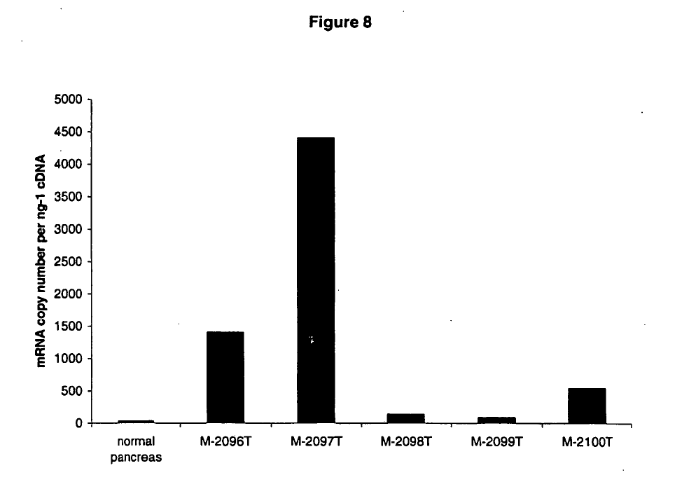

FIG. 8 shows the distribution of PTK7 mRNA in matched normal and tumour pancreatic tissues, mRNA levels were quantified by real time RT-PCR and are expressed as the number of copies ng⁻¹ cDNA.

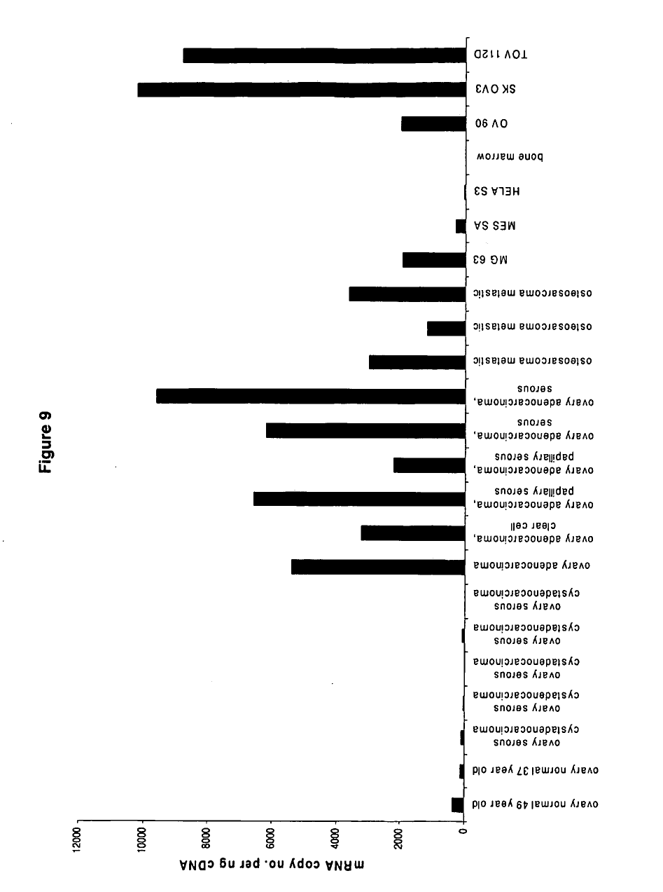

FIG. 9 shows the distribution of PTK7 mRNA in ovarian tumour and osteosarcoma samples; mRNA levels were quantified by real time RT-PCR and are expressed as the number of copies ng⁻¹ cDNA.

### EXAMPLE 1

### Isolation of PTK7 Protein from Lung and Liver Cell Lines

Proteins in lung and liver cancer cell line membranes were separated by SDS-PAGE and analysed.

Crude Fractionation of Lung and Liver Cancer Cell Lines

1a—Cell Culture

The lung cancer cell lines DMS144 and SHP-77 were cultured in EMEM plus 1% NEAA media, supplemented with 10% foetal calf serum, 2 mM glutamine, 1% penicillin and 1% streptomycin. The liver cancer cell line HepG2 was cultured in EMEM plus 1% NEAA media, supplemented with 10% foetal calf serum, 2 mM glutamine, 1% penicillin and 1% streptomycin. The cells were grown at 37° C. in a humidified atmosphere of 95% air and 5% carbon dioxide.

1b—Cell Fractionation and Plasma Membrane Generation

Purified membrane preparations were isolated from the cell lines. Adherent cells (2×10⁸) were washed three times with PBS and scraped using a plastic cell lifter. Cells were centrifuged at 1000×g for 5 min at 4° C. and the cell pellet was resuspended in homogenisation buffer (250 mM Sucrose, 10 mM HEPES, 1 mM EDTA, 1 mM Vanadate and 0.02% azide, protease inhibitors). Cells were fractionated using a ball bearing homogeniser (8.002 mm ball, HGM Lab equipment) until approx. 95% of cells were broken. Membranes were fractionated using the method described by Pasquali et al (Pasquali C. et al., 1999 J. Chromatography 722: pp 89-102). The fractionated cells were centrifuged at 3000×g for 10 min at 4° C. and the post-nuclear supernatant was layered onto a 60% sucrose cushion and centrifuged at 100 000×g for 45 min. The membranes were collected using a pasteur pipette and layered on a preformed 15 to 60% sucrose gradient and spun at 100 000×g for 17 hr. Proteins from the fractionated sucrose gradient were run on a 4-20% gel (Novex) and subject to Western blotting.

1c—Preparation of Plasma Membrane Fractions for 1D-Gel Analysis

Plasma membrane fractions that had transferrin immunoreactivity but no oxidoreductase II or calnexin immunoreactivity were pooled and represented the plasma membrane fraction. These sucrose fractions were pooled and diluted at least four times with 10 mM HEPES, 1 mM EDTA 1 mM Vanadate, 0.02% Azide. The diluted sucrose fraction was added to a SW40 or SW60 tube and centrifuged at 100 000×g for 45 min with slow acceleration and deceleration. The supernatant was removed from the membrane pellet and the pellet washed three times with PBS-CM. The membrane pellet was solubilised in 2% SDS in 63 mM Tris HCl, pH 7.4. A protein assay was performed followed by the addition of mercaptoethanol (2% final), glycerol (10%) and bromophenol blue (0.0025% final) was added. A final protein concentration of 1 microgram/microlitre was used for 1 D-gel loading.

1d—1D-Gel Technology

Protein or membrane pellets were solubilised in 1D-sample buffer (approximately 1 mg/ml) and the mixture heated to 95° C. for 5 min.

Samples were separated using 1 D-gel electrophoresis on pre-cast 8-16% gradient gels purchased from Bio-Rad (Bio-Rad Laboratories, Hemel Hempstead, UK). A sample containing 30-50 micrograms of the protein mixtures obtained from a detergent extract were applied to the stacking gel wells using a micro-pipette. A well containing molecular weight markers (10, 15, 25, 37, 50, 75, 100, 150 and 250 kDa) was included for calibration by interpolation of the separating gel after imaging. Separation of the proteins was performed by applying a current of 30 mA to the gel for approximately 5 hrs or until the bromophenol blue marker dye had reached the bottom of the gel.

After electrophoresis the gel plates were prised open, the gel placed in a tray of fixer (10% acetic acid, 40% ethanol, 50% water) and shaken overnight. The gel was then primed for 30 min by shaking in a primer solution (7.5% acetic acid, 0.05% SDS in Milli-Q water) followed by incubation with a fluorescent dye (0.06% OGS dye in 7.5% acetic acid) with shaking for 3 hrs. A preferred fluorescent dye is disclosed in U.S. Pat. No. 6,335,446. Sypro Red (Molecular Probes, Inc., Eugene, Oreg.) is a suitable alternative dye for this purpose.

A digital image of the stained gel was obtained by scanning on a Storm Scanner (Molecular Dynamics Inc, USA) in the blue fluorescence mode. The captured image was used to determine the area of the gel to excise for in-gel proteolysis.

1e—Recovery and Analysis of Selected Proteins

Each vertical lane of the gel was excised using either a stainless steel scalpel blade or a PEEK gel cutter (OGS) that cuts sequentially down the length of the gel lane with no attempt at collecting specific protein bands.

Proteins were processed using in-gel digestion with trypsin (Modified trypsin, Promega, Wis., USA) to generate tryptic digest peptides. Recovered samples were divided into two. Prior to MALDI analysis samples were desalted and concentrated using C18 Zip TiPS™ (Millipore, Bedford, Mass.). Samples for tandem mass spectrometry were purified using a nano LC system (LC Packings, Amsterdam, The Netherlands) incorporating C18 SPE material. Recovered peptide pools were analysed by MALDI-TOF-mass spectrometry (Voyager STR, Applied Biosystems, Framingham, Mass.) using a 337 nm wavelength laser for desorption and the reflectron mode of analysis. Pools were also analyzed by nano-LC tandem mass spectrometry (LC/MS/MS) using a Micromass Quadrupole Time-of-Flight (Q-TOF) mass spectrometer (Micromass, Altrincham, UK). For partial amino acid sequencing and identification of membrane proteins uninterpreted tandem mass spectra of tryptic peptides were searched against a database of public domain proteins constructed of protein entries in the non-redundant database held by the National Centre for Biotechnology Information (NCBI) which is accessible at http://www.ncbi.nlm.nih.gov/ using the SEQUEST search program (Eng et al., 1994, J. Am. Soc. Mass Spectrom. 5:976-989), version v.C.1. Criteria for database identification included: the cleavage specificity of trypsin; the detection of a suite of a, b and y ions in peptides returned from the database, and a mass increment for all Cys residues to account for carbamidomethylation. Following identification of proteins through spectral-spectral correlation using the SEQUEST program, masses detected in MALDI-TOF mass spectra were assigned to tryptic digest peptides within the proteins identified. In cases where no amino acid sequences could be identified through searching with uninterpreted MS/MS spectra of tryptic digest peptides using the SEQUEST program, tandem mass spectra of the peptides were interpreted manually, using methods known in the art. (In the case of interpretation of low-energy fragmentation mass spectra of peptide ions see Gaskell et al., 1992, Rapid Commun. Mass Spectrom. 6:658-662). The method described in WO 02/21139 was also used to interpret mass spectra.

Four tandem spectra (shown in bold and underlined in FIG. 1) and 16 mass matches (shown in bold in FIG. 1) were found to match the GenBank accession numbers AAC50484 and JC4593.

### EXAMPLE 2

### Normal Tissue Distribution and Disease Tissue Upregulation of PTK7 Using Quantitative RT-PCR (Taqman) Analysis

Real time RT-PCR was used to quantitatively measure PTK7 expression in a range of tumour tissues and matched controls. Ethical approval for the normal and tumour breast samples was obtained at surgery (University of Oxford, UK). The lung, pancreatic and kidney tumour samples were obtained from Clinomics Inc. and ovary and osteosarcoma tumour samples were obtained from Ardais Corp. (Lexington, Mass.). The primers used for PCR were as follows:

| | |
|---|---|
| Sense, | |
| 5′- cagccagaacttcaccttgagc - 3′, | (SEQ ID NO: 3) |
| Antisense, | |
| 5′- catgggagtctcatcctcaaag - 3′ | (SEQ ID NO: 4) |

Reactions containing 5 ng cDNA, SYBR green sequence detection reagents (PE Biosystems) and sense and antisense primers were assayed on an ABI7700 sequence detection system (PE Biosystems). The PCR conditions were 1 cycle at 50° C. for 2 min, 1 cycle at 95° C. for 10 min, and 40 cycles of 95° C. for 15 s, 60° C. for 1 min. The accumulation of PCR product was measured in real time as the increase in SYBR green fluorescence, and the data were analysed using the Sequence Detector program v1.6.3 (PE Biosystems). Standard curves relating initial template copy number to fluorescence and amplification cycle were generated using the amplified PCR product as a template, and were used to calculate PTK7 copy number in each sample.

Relatively low expression levels of PTK7 were seen in normal tissues (less 2000 copies per ng⁻¹ cDNA, FIG. 3).

In contrast, levels of PTK7 expression were greatly increased in breast tumour samples relative to their matched controls with 8/23 samples showing expression levels of greater than 2500 copies per ng-1 cDNA (FIG. 4). Furthermore, a large upregulation in PTK7 expression could be seen in 9/20 samples from patients with lymph node metastases (>50,000 copies per ng⁻¹ cDNA) compared to both normal tissue and tissue from patients with no lymph node metastases (FIG. 5).

The expression of PTK7 expression was examined in 7 lung tumour samples and an increased expression was seen in all seven samples relative to a control sample of unaffected lung (FIG. 6). PTK7 mRNA expression was also increased in 8/9 kidney tumour samples relative to either control kidney tissue or a matched control sample (FIG. 7), in 6/6 pancreatic tumour samples relative to normal tissue (FIG. 8), 6/11 ovarian cancer samples, in 3/3 osteosarcomas and in 3 ovarian cancer cell lines and in MG 63 osteosarcoma cells (FIG. 9).

### EXAMPLE 3

### Generation of Stable HEK 293 and CHO-K1 Cells Overexpressing PTK7

HEK 293 cells (primary human embryo kidney cells, ATCC no: CRL-1537) and CHOK1 cells (ATTC no: CCL 61) were grown in Dulbecco's medium NUT mix F12, 10% Fetal Calf serum, 2 mM glutamine. PTK7 (accession number: U40271) was cloned into pcDNA3.1 neomycin vector (Invitrogen) and this vector was transfected into pcDNA3.1 cells (Gene Juice, Novagen).

A pool of HEK293 cells expressing full-length PTK7 was selected for growth in antibiotic-containing medium (0.2 mg/ml G418, Sigma). CHO-K1 cells expressing full-length PTK7 were dilution cloned and selected for growth in antibiotic-containing medium (0.2 mg/ml G418). Two CHO-K1 clones, A3 and A6, were selected for further assessment.

### EXAMPLE 4

### Detection of PTK7 In Cancer Cell Lines

A polyclonal antibody was raised against PTK7 (CovalAb, Lyon, France). The antibody was raised in rabbits immunized with two specific peptides whose sequences were chosen for synthesis based on plots of hydrophobicity, antigenicity, surface probability, and low identity to other known protein family members. Peptides were synthesized using Fmoc chemistry with a cysteine residue added to the end to enable specific thiol reactive coupling of Keyhole limpet Hemocyanin prior to immunization. The PTK7 peptides used were: KGKDRILDPTKLGP (SEQ ID NO: 5, peptide 010-II, extracellular epitope) and ISKSKDEKLKSQPL (SEQ ID NO: 6, peptide 011-II, intracellular epitope).

Western blotting with the antisera (peptide 010-II) recognized a band of approx. 160 KDa in HEK 293 and CHO-K1 cell lysate overexpressing PTK7 but not in the parental HEK 293 cells. This recombinant protein also expressed a V5 tag which is recognized by a specific monoclonal antibody (Invitrogen). Western Blotting with the anti-V5 tag antibody also recognized a single band of approx. 160 kDa in HEK 293 cell lysate expressing HEK 2.

This antisera also recognized a protein of identical molecular weight in the cancer cell lines BT474 (breast carcinoma cell line, ATCC no: HTB 20 ), BT20 (breast carcinoma cell line, ATCC no: HTB 19), PANC-1 (pancreatic carcinoma cell line, ATCC no: CRL-1469), T47D (breast carcinoma cell line, HTB 133) and SK MEL 5 (malignant melanoma cell line, ATCC no: HTB-70) and SaOS-2 (osteogenic sarcoma cell line, ATCC no: HTB 85).

### EXAMPLE 5

### Cellular Localisation of PTK7 in Cancer Cell Lines

Fluorescent immunocytochemistry was used to assess the cellular localisation of recombinant or endogenous PTK7 in cell lines.

Cancer cell lines, and transfected and non-transfected HEK293 cells and CHO-K1 cells, were seeded onto 8-well chamber slides (Nalge, Nunc) at a density of 6×10⁴ cells per well. After 24 hours of incubation at 37° C. and 5% CO2 the media was removed from the slides and the plastic housing of the chambers was also removed. The slides were washed once in PBS in a Coplin jar for five minutes. Excess PBS was removed from the slides and then they were placed in a 10% formalin solution for fifteen minutes. Following this incubation, excess liquid was removed from the slides and then the slides were laid flat in a humidifier chamber and incubated for 1 hr with 500 μl of primary antibody appropriately diluted in PBS 1% (w/v) BSA (1 μg/ml of rabbit anti-PTK7 antisera 010-II or 1 μg/ml rabbit gamma globulin (Sigma). Slides were then washed three times for five minutes per wash in PBS. The slides were incubated for 1 hr in a humidifier chamber with 500 μl of biotin-SP-conjugated AffiniPure donkey anti-rabbit antibody, (Jackson ImmunoResearch) diluted 1:200 in PBS 1% (w/v) BSA. Following this incubation the slides were washed three times for five minutes per wash in PBS and then incubated for 1 hr in a humidifier chamber with 500 μl of Cy3-conjugated-Extravidin (Sigma) diluted 1:700 in PBS 1% (w/v) BSA. Following this incubation the slides were washed four times for five minutes per wash in PBS. Slides were then incubated for 30 sec with 2 μg/ml BisBenzimide (Sigma) and then washed for 2 min in PBS. Slides were mounted with a coverslip in fluorescent enhancing media (Dako Ltd.) and then images were captured by digital camera DC300F attached to a DMIRE2 fluorescence microscope (Leica Microsystems (UK) Ltd.).

The resulting images demonstrated that PTK7 is highly expressed on the plasma membrane of PTK7-transfected HEK293 and CHO cells. This staining is highly specific to the PTK7 transfected cells since staining of non-transfected HEK293 and CHO-1 cells with anti-PTK7 polyclonal antibody reveals very low levels of fluorescence. Control rabbit IgG staining of PTK7 transfected cells gives a very low level of background fluorescence.

Additionally, the cell lines Panc1, SaOS-2, MDA-MB-453 and MDA-MB-361 all showed clear membrane localised endogenous PTK7.

### EXAMPLE 6

### PTK7 Immunohistochemistry

Immunohistochemical analysis was carried out on formalin-fixed paraffin-embedded tissue microarrays containing 1 mm sections of breast carcinoma tissue from 55 donors, prostate carcinoma tissue from 50 donors, lung carcinoma tissue from 50 donors as well as HDCS containing 250 sections of various normal and carcinoma tissues (Clinomics Laboratories Inc., 165 Tor Court, Pittsfield, Mass. 01201).

Slides were deparafinised by 2×5 min washes in xylene then rehydrated through successive graded ethanol solutions and washed for 5 min in PBS. Antigen retrieval was achieved by immersing the slides in 0.01 M citrate buffer (pH 6) and microwaving for 10 min at full power (950 W). In addition, detection with the antibody was improved by protease treatment of the tissue with Autozyme (AbCam) for 10 min at room temperature. The tissue was blocked in 10% donkey serum/PBS for 1 hr before addition of 1.5 μg/ml antisera 010-II (in 2.5% donkey serum/PBS). Following 3 washes in PBS the tissue sections were incubated with biotin-conjugated secondary antibodies (Biotin-SP-conjugated AffiniPure Donkey anti-guinea pig, Jackson ImmunoResearch) diluted at 1:200 (2.5 μg/ml in 2.5% donkey serum/PBS) for 1 h. Slides were washed 3 times in PBS and the tissue incubated with Streptavidin-HRP (Jackson ImmunoResearch) diluted 1:100 (5 μg/ml in 2.5% donkey serum/PBS), followed by 3×5 min washes in PBS. Antibody signal was detected using DAB substrate solution (Dako Ltd.) according to the manufacturers' instructions. An adjacent tissue array was counterstained for hematoxylin and eosin (Dako Ltd.) and images were captured by a digital camera attached to a light microscope.

Immunohistochemical analysis of PTK7 on the breast tumour microarray demonstrated that PTK7 was present in 35-40% of the breast carcinoma tissue sections examined. Only tumour cells displayed positive PTK7 staining, the stroma and adjacent normal tissue did not stain. All tumour cells in a section had positive PTK7 immunoreactivity.

In the patient matched primary breast carcinoma tissue and lymph node metastasis sections, 5/5 metastatic lymph nodes and 4/4 matched primary breast carcinoma tissue sections had PTK7 immunoreactive staining. Expression of PTK7 was generally restricted to the cancerous epithelial cells of the tumour tissue and membrane localisation could be detected.

Tumour cell specific positive staining for PTK7 was also seen in colon cancer (38 of 55 samples), pancreatic cancer (1 of 7 samples), kidney cancer (1 of 7 samples, where the staining appeared to be mainly cytosolic) and bladder cancer (1 of 7 samples).

PTK7 immunoreactive staining was not detected in prostate cancer (16 samples), liver cancer (7 samples), stomach cancer (7 samples), endometrial cancer (7 samples), thyroid cancer (10 samples), melanoma (5 samples), lymphoma (14 samples).

Little or no PTK7 immunoreactive staining could be detected in the following normal tissues: prostate, liver, kidney, thyroid, spleen, lung, colon, lymph node, pancreas, heart, brain, adrenal, testicle, ovary. The PTK7 immunoreactive staining was not distinguishable from staining observed with rabbit IgG control antibody.

### EXAMPLE 7

### Induction of Colony Formation by PTK7

HEK293 cells expressing recombinant PTK7 in HEK293 cells or HEK293 cells transfected with empty pcDNA3.1 vector (vector control cells) were assessed for colony forming ability.

6-well tissue culture plates were coated in a base layer of 0.6% agar and left to set for 10 minutes at room temperature. Cells were seeded in the top layer of agarose (0.4%) at a final concentration of 5000 cells/ml (HEK 293 pcDNA3.1 vector control cells, HEK 293 parental cells and PTK7 expressing HEK 293 cells) and 2 ml of this added to each well to give a final count of 10,000 cells/well (in triplicate). Plates were incubated at 37° C. 5% CO₂ for 10-14 days. The number of colonies comprising of more than one cell was assessed for each condition. PTK7 transfected HEK293 cells formed many large colonies whereas control transfected and HEK293 parentals were unable to establish themselves in the soft agar and no colonies were formed.

This data suggests that PTK7 is increased in a selection of carcinomas including breast cancer, lung cancer, kidney cancer and pancreatic cancer at the protein and the mRNA level. In addition, a large increase in PTK7 expression is seen in 45% of samples from breast cancer patients with lymph node metastases relative to both normal tissue and samples from breast cancer patients with no lymph node metastases, indicating that PTK7 is of utility as a carcinoma target, in particular, a breast cancer target. PTK7 appears to induce dramatic colony formation activity in HEK293 cells and as such suggests PTK7 to be a strong factor in anchorage independence, a common trait of transformed cells.

1. A method for the treatment and/or prophylaxis of carcinoma comprising administering a therapeutically effective amount of an agent which interacts with or modulates the expression or activity of a PTK7 polypeptide.

2. The method of claim 1, wherein the PTK7 polypeptide:
(a) comprises or consists of the amino acid sequence of SEQ ID NO:1; or
(b) is a derivative having one or more amino acid substitutions, modifications, deletions or insertions relative to the amino acid sequence of SEQ ID NO:1 which retains the activity of the PTK7 polypeptide.

3. The method of claim 1, wherein the agent is an antibody, functionally-active fragment, derivative or analogue thereof.

4. The method of claim 3, wherein the antibody is monoclonal, polyclonal, chimeric, humanised or bispecific, or is conjugated to a therapeutic moiety, detectable label, second antibody or a fragment thereof, a cytotoxic agent or cytokine.

5. The method of claim 2, wherein the agent is an antibody, functionally-active fragment, derivative or analogue thereof.

6. The method of claim 5, wherein the antibody is monoclonal, polyclonal, chimeric, humanised or bispecific, or is conjugated to a therapeutic moiety, detectable label, second antibody or a fragment thereof, a cytotoxic agent or cytokine.

7. The method as claimed in claim 1, wherein the composition is a vaccine.

8. A method of screening for anti-carcinoma agents that interact with a PTK7 polypeptide, said method comprising:
(a) contacting said polypeptide with a candidate agent; and
(b) determining whether or not the candidate agent interacts with said polypeptide.

9. The method according to claim 8, wherein the determination of an interaction between the candidate agent and PTK7 polypeptide comprises quantitatively detecting binding of the candidate agent and said polypeptide.

10. A method of screening for anti-carcinoma agents that modulate the expression or activity of a PTK7 polypeptide comprising:
(i) comparing the expression or activity of said polypeptide in the presence of a candidate agent with the expression or activity of said polypeptide in the absence of the candidate agent or in the presence of a control agent; and
(ii) determining whether the candidate agent causes the expression or activity of said polypeptide to change.

11. The method of claim 10, wherein the expression or activity of said polypeptide, is compared with a predetermined reference range.

12. The method of claim 10, wherein part (ii) additionally comprises selecting an agent which interacts with or modulates the expression or activity of said polypeptide for further testing, or therapeutic or prophylactic use as an anti-carcinoma agent.

13. An agent identified by the method of claim 10, which interacts with or causes the expression or activity of said polypeptide to change.

14. (canceled)

15. A method of screening for and/or diagnosis or prognosis of carcinoma in a subject, and/or monitoring the effectiveness of carcinoma therapy, which comprises the step of detecting and/or quantifying, in a biological sample obtained from said subject, a PTK7 polypeptide.

16. The method of claim 15, wherein the level of said polypeptide is compared to a previously determined reference range or control.

17. The method according to claim 15, wherein the step of detecting comprises:
(a) contacting the sample with a capture reagent that is specific for a PTK7 polypeptide; and
(b) detecting whether binding has occurred between the capture reagent and said polypeptide in the sample.

18. The method according to claim 17, wherein step (b) comprises detecting the captured polypeptide using a directly or indirectly labelled detection reagent.

19. The method according to claim 17, wherein the capture reagent is immobilised on a solid phase.

20. The method according to of claim 8, wherein the polypeptide is detected and/or quantified using an antibody that specifically binds to a PTK7 polypeptide.

21. The method of claim 20, wherein the antibody is conjugated to a detectable label, or a second antibody or a fragment thereof.

22. A diagnostic kit comprising a capture reagent specific for a PTK7 polypeptide, reagents and instructions for use.

23. The method of claim 1, wherein the carcinoma is breast, ovarian, pancreatic, lung, bladder or kidney cancer or osteosarcoma.

24. The method of claim 1, wherein the carcinoma is breast cancer.

25. The method of claim 11, wherein part (ii) additionally comprises selecting an agent which interacts with or modulates the expression or activity of said polypeptide for further testing, or therapeutic or prophylactic use as an anti-carcinoma agent.

26. An agent identified by the method claim 11, which interacts with or causes the expression or activity of said polypeptide to change.

27. An agent identified by the method claim 12, which interacts with or causes the expression or activity of said polypeptide to change.

28. An agent identified by the method claim 25, which interacts with or causes the expression or activity of said polypeptide to change.

29. The method according to claim 16, wherein the step of detecting comprises:
(a) contacting the sample with a capture reagent that is specific for a PTK7 polypeptide; and
(b) detecting whether binding has occurred between the capture reagent and said polypeptide in the sample.

30. The method according to claim 29, wherein step (b) comprises detecting the captured polypeptide using a directly or indirectly labelled detection reagent.

31. The method according to claim 18, wherein the capture reagent is immobilised on a solid phase.

32. The method according to claim 30, wherein the capture reagent is immobilised on a solid phase.


SEQUENCE LISTING (6 sequences)

SEQ ID NO: 1 - protein, 1070 aa, Homo Sapiens [sequence omitted]

SEQ ID NO: 2 - DNA, 4191 bp, Homo Sapiens
cgcctcgggacgcctcggggtcgggctccggctgcggctgctgctgcggcgcccgcgctccggtgcgctccgcctcctgtgcccgccgcggagcgcagtctgcgcgcccgccgtgcgccctcagctccttttcctgagcccgccgcgatgggagctgcgcggggatccccggccagaccccgccggttgcctctgctcagcgtcctgctgctgccgctgctgggcggtacccagacagccattgtcttcatcaagcagccgtcctcccaggatgcactgcaggggcgccgggcgctgcttcgctgtgaggttgaggctccgggcccggtacatgtgtactggctgctcgatggggcccctgtccaggacacggagcggcgtttcgcccagggcagcagcctgagctttgcagctgtggaccggctgcaggactctggcaccttccagtgtgtggctcgggatgatgtcactggagaagaagcccgcagtgccaacgcctccttcaacatcaaatggattgaggcaggtcctgtggtcctgaagcatccagcctcggaagctgagatccagccacagacccaggtcacacttcgttgccacattgatgggcaccctcggcccacctaccaatggttccgagatgggacccccctttctgatggtcagagcaaccacacagtcagcagcaaggagcggaacctgacgctccggccagctggtcctgagcatagtgggctgtattcctgctgcgcccacagtgcttttggccaggcttgcagcagccagaacttcaccttgagcattgctgatgaaagctttgccagggtggtgctggcaccccaggacgtggtagtagcgaggtatgaggaggccatgttccattgccagttctcagcccagccacccccgagcctgcagtggctctttgaggatgagactcccatcactaaccgcagtcgccccccacacctccgcagagccacagtgtttgccaacgggtctctgctgctgacccaggtccggccacgcaatgcagggatctaccgctgcattggccaggggcagaggggcccacccatcatcctggaagccacacttcacctagcagagattgaagacatgccgctatttgagccacgggtgtttacagctggcagcgaggagcgtgtgacctgccttccccccaagggtctgccagagcccagcgtgtggtgggagcacgcgggagtccggctgcccacccatggcagggtctaccagaagggccacgagctggtgttggccaatattgctgaaagtgatgctggtgtctacacctgccacgcggccaacctggctggtcagcggagacaggatgtcaacatcactgtggccactgtgccctcctggctgaagaagccccaagacagccagctggaggagggcaaacccggctacttggattgcctgacccaggccacaccaaaacctacagttgtctggtacagaaaccagatgctcatctcagaggactcacggttcgaggtcttcaagaatgggaccttgcgcatcaacagcgtggaggtgtatgatgggacatggtaccgttgtatgagcagcaccccagccggcagcatcgaggcgcaagcccgtgtccaagtgctggaaaagctcaagttcacaccaccaccccagccacagcagtgcatggagtttgacaaggaggccacggtgccctgttcagccacaggccgagagaagcccactattaagtgggaacgggcagatgggagcagcctcccagagtgggtgacagacaacgctgggaccctgcattttgcccgggtgactcgagatgacgctggcaactacacttgcattgcctccaacgggccgcagggccagattcgtgcccatgtccagctcactgtggcagtttttatcaccttcaaagtggaaccagagcgtacgactgtgtaccagggccacacagccctactgcagtgcgaggcccagggggaccccaagccgctgattcagtggaaaggcaaggaccgcatcctggaccccaccaagctgggacccaggatgcacatcttccagaatggctccctggtgatccatgacgtggcccctgaggactcaggccgctacacctgcattgcaggcaacagctgcaacatcaagcacacggaggcccccctctatgtcgtggacaagcctgtgccggaggagtcggagggccctggcagccctcccccctacaagatgatccagaccattgggttgtcggtgggtgccgctgtggcctacatcattgccgtgctgggcctcatgttctactgcaagaagcgctgcaaagccaagcggctgcagaagcagcccgagggcgaggagccagagatggaatgcctcaacggtgggcctttgcagaacgggcagccctcagcagagatccaagaagaagtggccttgaccagcttgggctccggccccgcggccaccaacaaacgccacagcacaagtgataagatgcacttcccacggtctagcctgcagcccatcaccacgctggggaagagtgagtttggggaggtgttcctggcaaaggctcagggcttggaggagggagtggcagagaccctggtacttgtgaagagcctgcagagcaaggatgagcagcagcagctggacttccggagggagttggagatgtttgggaagctgaaccacgccaacgtggtgcggctcctggggctgtgccgggaggctgagccccactacatggtgctggaatatgtggatctgggagacctcaagcagttcctgaggatttccaagagcaaggatgaaaaattgaagtcacagcccctcagcaccaagcagaaggtggccctatgcacccaggtagccctgggcatggagcacctgtccaacaaccgctttgtgcataaggacttggctgcgcgtaactgcctggtcagtgcccagagacaagtgaaggtgtctgccctgggcctcagcaaggatgtgtacaacagtgagtactaccacttccgccaggcctgggtgccgctgcgctggatgtcccccgaggccatcctggagggtgacttctctaccaagtctgatgtctgggccttcggtgtgctgatgtgggaagtgtttacacatggagagatgccccatggtgggcaggcagatgatgaagtactggcagatttgcaggctgggaaggctagacttcctcagcccgagggctgcccttccaaactctatcggctgatgcagcgctgctgggccctcagccccaaggaccggccctccttcagtgagattgccagcgccctgggagacagcaccgtggacagcaagccgtgaggagggagcccgctcaggatggcctgggcaggggaggacatctctagagggaagctcacagcatgatgggcaagatccctgtcctcctgggccctgaggcccctgccctagtgcaacaggcattgctgaggtctgagcagggcctggcctttcctcctcttcctcaccctcatcctttgggaggctgacttggacccaaactgggcgactagggctttgagctgggcagtttcccctgccacctcttcctctatcagggacagtgtgggtgccacaggtaaccccaatttctggccttcaacttctccccttgaccgggtccaactctgccactcatctgccaactttgcctggggagggctaggcttgggatgagctgggtttgtggggagttccttaatattctcaagttctgggcacacagggttaatgagtctcttcgcccactggtcccacttgggggtctagaccaggattatagaggacacagcaagtgagtcctccccactctgggcttgtgcacactgacccagacccacgtcttccccacccttctctcctttcctcatcctaagtgcctggcagatgaaggagttttcaggagcttttgacactatataaaccgccctttttgtatgcaccacgggcggcttttatatgtaattgcagcgtggggtgggtgggcatgggaggtaggggtgggccctggagatgaggagggtgggccatccttaccccacacttttattgttgtcgttttttgtttgttttgtttttttgtttttgtttttgtttttacactcgctgctctcaataaataagcctttttta

SEQ ID NO: 3 - DNA, 22 bp, Homo Sapiens
cagccagaacttcaccttgagc

SEQ ID NO: 4 - DNA, 22 bp, Homo Sapiens
catgggagtctcatcctcaaag

SEQ ID NO: 5 - protein, 14 aa, Homo Sapiens
KGKDRILDPTKLGP

SEQ ID NO: 6 - protein, 14 aa, Homo Sapiens
ISKSKDEKLKSQPL